SEQUENCE LISTING (4 sequences)

SEQ ID NO: 1 - protein, 392 aa, Bacillus subtilis
MKKYHISMINIPAYGHVNPTLALVEKLCEKGHRVTYATTEEFAPAVQQAGGEALIYHTSLNIDPKQIREMMEKNDAPLSLLKESLSILPQLEELYKDDQPDLIIYDFVALAGKLFAEKLNVPVIKLCSSYAQNESFQLGNEDMLKKIREAEAEFKAYLEQEKLPAVSFEQLAVPEALNIVFMPKSFQIQHETFDDRFCFVGPSLGERKEKESLLIDKDDRPLMLISLGTAFNAWPEFYKMCIKAFRDSSWQVIMSVGKTIDPESLEDIPANFTIRQSVPQLEVLEKADLFISHGGMNSTMEAMNAGVPLVVIPQMYEQELTANRVDELGLGVYLPKEEVTVSSLQEAVQAVSSDQELLSRVKNMQKDVKEAGGAERAAAEIEAFMKKSAVPQ

SEQ ID NO: 2 - protein, 400 aa, Bacillus cereus
MANVLVINFPGEGHINPTLAIVSELIRRGETVVSYCIEDYRKKIEATGAQFRVFENFLSQINIMERVNEGGSPLTMLSHMMEASERIVTQIVEETKGEKYDYLIYDNHFPVGRIIANVLKLPSVSSCTTFAFNQYITFNDEHESREVDETNPLYQSCLAGMEKWNKQYGMKCNSMYDIMNHPGDITIVYTSKEYQPRSDVFDESYKFVGPSIATRKEVGSFPMEDLKDEKLIFISMGTVFNEQPELYEKCFEAFKDVEATVVLVVGKKINISQFENIPNNFKLYNYVPQLELLQYADVFVTHGGMNSSSEALYYGVPLVVIPVTGDQPLVAKRVNEVGAGIRLNRKELTSEMLRESVKKVMDDVTFKEKSRKVGESLRNAGGYNRAVDEILKMNSYSKLK

SEQ ID NO: 3 - protein, 394 aa, Bacillus methylotrophicus
MRKTHIAIINVAAHGHVNPTLPVAEELVNRGYKVTFATTEEFEASVAKTGAIPVLYRTSIKADPETIKERVNKNDAFVMFLEEAVEVLPQLEELYKDDLPDIVLFDFLALAGRLFADNGGIPAVKFCPSYAMNEYFQLGRDEETLEAAKQAIEEYQEQIENEQLKRMTMEEFFMPEKLNIVFMPRAFQPKQETFDERFCFVGPSLGERTNTGSLEIDAADDRPLMLISLGTAFNAWPEFYRMCIDAFRDSDWRVIMSVGTTIDPESFSDVPDHFIIRQHVPQLDVLEKAKLFVSHGGMNSTMEAMNAGVPLVVVPQMHEQEVTAKRVDELGLGVHLPPKEVTVARLQKDVQNVYGDKNILSRVKSMQQKIEETGGPKQAVCAIEEFLKTAAVEN

SEQ ID NO: 4 - protein, 396 aa, Bacillus licheniformis
MGHKHIAIFNIPAHGHINPTLALTASLVKRGYRVTYPVTDEFVKAVEETGAEPLNYRSTLNIDPQQIRELMKNKKDMSQAPLMFIKEMEEVLPQLEALYENDKPDLILFDFMAMAGKLLAEKFGIEAVRLCSTYAQNEHFTFRSISEEFKIELTPEQEDALKNSNLPSFNFEDMFEPAKLNIVFMPRAFQPYGETFDERFSFVGPSLAKRKFQEKETPIISDSGRPVMLISLGTAFNAWPEFYHMCIEAFRDTKWQVIMAVGTTIDPESFDDIPENFSIHQRVPQLEILKKAELFITHGGMNSTMEGLNAGVPLVAVPQMPEQEITARRVEELGLGKHLQPEDTTAASLREAVSQTDGDPHVLKRIQDMQKHIKQAGGAEKAADEIEAFLAPAGVK

(19) United States
(12) Patent Application Publication
(10) Pub. No.: US 2023/0124589 A1
(43) Pub. Date: Apr. 20, 2023

(54) Ivacaftor Glycosides, Methods Of Making, And Uses Thereof In Treating Cystic Fibrosis

(71) Applicant: DoubleRainbow Biosciences Inc., Lexington, MA (US)

(72) Inventors: Sheng Ding, Bethany, CT (US); Yasmin-Pei Kamal Chau, Cambridge, MA (US); Jacob Donald Stanley Wirth, Waltham, MA (US); Tian Xu, Guilford, CT (US); Jing-Ke Weng, Belmont, MA (US)

(21) Appl. No.: 17/906,396

(22) PCT Filed: Mar. 15, 2021
(86) PCT No.: PCT/US2021/022416

§ 371 Date: Sep. 15, 2022

Related U.S. Application Data
(60) Provisional application No. 62/990,101, filed on Mar. 16, 2020.

(51) Int. Cl. C12P 19/60; C12N 9/10; A61K 47/61; A61K 47/54

(52) U.S. Cl. CPC C12P 19/60; C12N 9/1051; A61K 47/61; A61K 47/549; C12Y 204/01

(57) ABSTRACT

Ivacaftor glycosides and methods of making ivacaftor glycosides are disclosed. Glycosyl transferases catalyze addition of one or more monosaccharides to ivacaftor to yield ivacaftor glycosides. Suitable monosaccharides can be in the L- or D-configuration and typically have 5, 6, or 7 carbons. Suitable monosaccharides include allose, apiose, arabinose, fructose, fucitol, fucose, galactose, glucose, glucuronic acid, mannose, A-acetylglucosamine, rhamnose, or xylose. Uridine diphosphate glycosyl transferases can catalyze formation of either an alpha or beta glycosidic bond.

### RELATED APPLICATION

This application claims the benefit of U.S. Provisional Application No. 62/990,101, filed on Mar. 16, 2020. The entire teachings of the above application are incorporated herein by reference.

### INCORPORATION BY REFERENCE OF MATERIAL IN ASCII TEXT FILE

This application incorporates by reference the Sequence Listing contained in the following ASCII text file being submitted concurrently herewith:
a) File name: 57671000001SequenceListing.txt; created Mar. 15, 2021, 14 KB in size.

### BACKGROUND

Cystic fibrosis (CF) is a progressive genetic disease that affects approximately 30,000 people in the United States and approximately 70,000 people worldwide (“About Cystic Fibrosis” n.d.). Approximately 1,000 people are newly diagnosed with CF each year. 75% of CF diagnosis occurs by the age of 2 as a result of newborn genetic testing, although less severe forms of the disease can result in diagnosis in adulthood (Desai et al. 2018). While the survival outlook for CF patients has markedly improved over the past decade with patients predicted to survive well into their 30's and 40's (Cystic Fibrosis Foundation 2018), CF is still a progressive disease that worsens over time and requires lifelong medical and dietary management of many disease-related symptoms.

### SUMMARY

Described herein are ivacaftor derivatives containing specific monosaccharide(s) or oligosaccharides(s) and methods of making these molecules utilizing enzyme catalysis. Compared to ivacaftor, the ivacaftor glycosides exhibit increased water solubility, which may contribute to improved pharmacokinetic and/or pharmacodynamic profiles. The compounds may act as prodrugs of ivacaftor. The compounds may exhibit improvements in potency towards modulating the activity of the cystic fibrosis transmembrane conductance regulator (CFTR) protein. The compounds may exhibit enhanced therapeutic effects on cystic fibrosis and CFTR-related diseases.

Described herein are compounds represented by the following structural formula:

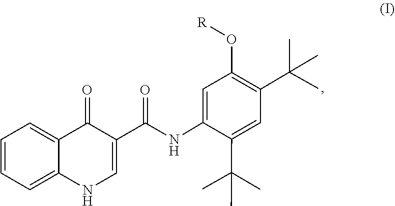

or a pharmaceutically acceptable salt thereof, wherein R is a monosaccharide, a disaccharide, a trisaccharide, or an oligosaccharide having 4 to 10 monosaccharides.

Described herein are pharmaceutical compositions that include an ivacaftor glycoside, or a pharmaceutically acceptable salt thereof, and a pharmaceutically acceptable carrier or adjuvant.

Described herein are methods of making an ivacaftor glycoside. The methods include: a) providing a reaction mixture; and b) allowing the reaction mixture to convert ivacaftor to a monosaccharide, a disaccharide, a trisaccharide, or an oligosaccharide of ivacaftor. The reaction mixture can include a compound having the following structural formula:

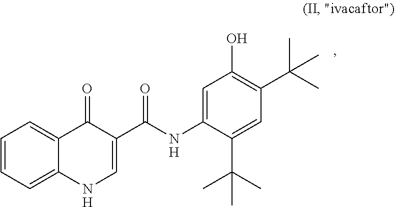

a uridine diphosphate glycosyltransferase (UGT); and uridine diphosphate-monosaccharide.

In some embodiments, R is a monosaccharide. In some embodiments, the monosaccharide is a pentose monosaccharide, hexose monosaccharide, or heptose monosaccharide.

In some embodiments, R is allose, apiose, arabinose, fructose, fucitol, fucose, galactose, glucose, glucuronic acid, mannose, N-acetylglucosamine, N-acetylgalactosamine, rhamnose, or xylose. In some embodiments, R is glucosamine, galactosamine, mannosamine, 5-thio-D-glucose, nojirimycin, deoxynojirimycin, 1,5-anhydro-D-sorbitol, 2,5-anhydro-D-mannitol, 2-deoxy-D-galactose, 2-deoxy-D-glucose, 3-deoxy-D-glucose, arabinitol, galactitol, glucitol, iditol, lyxose, mannitol, L-rhamnitol, 2-deoxy-D-ribose, ribose, ribitol, ribulose, xylulose, altrose, gulose, idose, levulose, psicose, sorbose, tagatose, talose, galactal, glucal, fucal, rhamnal, arabinal, xylal, 3,4-di-O-acetyl-L-fucal, 3,4-di-O-acetyl-L-rhamnal, 3,4-di-O-acetyl-D-arabinal, 3,4-di-O-acetyl-D-xylal, valienamine, validamine, valiolamine, valienol, valienone, galacturonic acid, mannuronic acid, N-acetylneuraminic acid, N-acetylmuramic acid, gluconic acid D-lactone, galactonic acid gamma-lactone, galactonic acid delta-lactone, mannonic acid gamma-lactone, D-altro-heptulose, D-manno-heptulose, D-glycero-D-manno-heptose, D-glycero-D-gluco-heptose, D-allo-heptulose, D-altro-3-heptulose, D-glycero-D-manno-heptitol, or D-glycero-D-altro-heptitol.

In some embodiments, R is a disaccharide. In some embodiments, R is a disaccharide of two glucose molecules. In some embodiments, R is a disaccharide of two galactose molecules. In some embodiments, R is a disaccharide of two xylose molecules. For any of the foregoing disaccharides, the disaccharide molecules can be bonded by a 1→2 glycosidic bond.

In some embodiments, R is a trisaccharide. In some embodiments, R is a trisaccharide of three glucose molecules. In some embodiments, R is a trisaccharide of three galactose molecules. In some embodiments, R is a trisaccharide of three xylose molecules. For any of the foregoing trisaccharides, the trisaccharide molecules can be bonded by a 1→2 glycosidic bond and by a 1→4 glycosidic bond.

In some embodiments, the UGT includes an amino acid sequence that is at least 95% similar to SEQ ID NO: 1. In some embodiments, the UGT includes an amino acid sequence that is at least 80% similar to a region from V278 to Q318 of SEQ ID NO: 1. In some embodiments, the UGT includes an amino acid sequence that is: at least 90% similar to a region from 167 to D75 of SEQ ID NO: 1; at least 90% similar to a region from D106 to L114 of SEQ ID NO: 1; at least 90% similar to a region from C127 to S129 of SEQ ID NO: 1; and at least 80% similar to a region from V278 to Q318 of SEQ ID NO: 1.

In some embodiments, the UGT includes an amino acid sequence that is at least 95% similar to SEQ ID NO: 2. In some embodiments, the UGT includes an amino acid sequence that is at least 80% similar to a region from V287 to Q327 of SEQ ID NO: 2. In some embodiments, the UGT includes an amino acid sequence that is: at least 90% similar to a region from 163 to G70 of SEQ ID NO: 2; at least 90% similar to a region from D106 to 1114 of SEQ ID NO: 2; at least 90% similar to a region from C127 to T129 of SEQ ID NO: 2; and at least 80% similar to a region from V287 to Q327 of SEQ ID NO: 2.

In some embodiments, the UGT includes an amino acid sequence that is at least 95% similar to SEQ ID NO: 3. In some embodiments, the UGT includes an amino acid sequence that is at least 80% similar to a region from V280 to Q320 of SEQ ID NO: 3. In some embodiments, the UGT includes an amino acid sequence that is: at least 90% similar to a region from 167 to D75 of SEQ ID NO: 3; at least 90% similar to a region from D106 to L114 of SEQ ID NO: 3; at least 90% similar to a region from C127 to S129 of SEQ ID NO: 3; and at least 80% similar to a region from V280 to Q320 of SEQ ID NO: 3.

In some embodiments, the UGT includes an amino acid sequence that is at least 95% similar to SEQ ID NO: 4. In some embodiments, the UGT includes an amino acid sequence that is at least 80% identical to a region from V283 to Q323 of SEQ ID NO: 4. In some embodiments, the UGT includes an amino acid sequence that is: at least 90% similar to a region from 167 to Q79 of SEQ ID NO: 4; at least 90% similar to a region from D110 to L118 of SEQ ID NO: 4; at least 90% similar to a region from C131 to T133 of SEQ ID NO: 4; and at least 80% similar to a region from V283 to Q323 of SEQ ID NO: 4.

In some embodiments, the uridine diphosphate-monosaccharide is uridine diphosphate-glucose (“UDP-glucose”), uridine diphosphate-galactose (“UDP-galactose”), uridine diphosphate-xylose (“UDP-xylose”), or uridine diphosphate-N-acetylglucosamine (“UDP-N-acetylglucosamine”).

Described herein are methods of treating cystic fibrosis or a cystic fibrosis transmembrane conductance regulator related disease. The method can include administering to a patient in need thereof a therapeutically effective amount of a compound having the following structural formula:

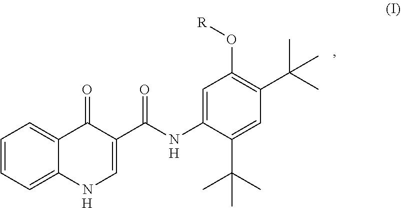

or a pharmaceutically acceptable salt thereof, wherein R is a monosaccharide, a disaccharide, a trisaccharide, or an oligosaccharide comprising 4 to 10 monosaccharides.

In some embodiments, the method further includes administering one or more of lumacaftor, tezacaftor, and elexacaftor to the patient.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 shows HPLC chromatograms of the UGT screen results using cell lysates from SEQ ID NO: 2 (2: top chromatogram) and empty vector only control (1: bottom chromatogram) when ivacaftor was used as substrate and UDP-glucose was used as the sugar donor. The two extra peaks present in the chromatogram of SEQ ID NO: 2 were glycosylated products ivacaftor-17-O-D-glucoside (chromatogram peak a) and ivacaftor-17-di-O-D-glucoside (chromatogram peak b).

FIG. 2 shows HPLC chromatograms of ivacaftor glycosides produced using the purified recombinant glycosyltransferase SEQ ID NO: 2 after an additional HPLC fractionation step to separate each ivacaftor glycoside. Chromatograms show purified ivacaftor-17-tri-O-D-glucoside (4: top chromatogram), ivacaftor-17-di-O-D-glucoside (3: second from the top chromatogram), ivacaftor-17-O-D-glucoside (2: third from the top chromatogram), and the substrate ivacaftor (1: bottom chromatogram). Labeled peaks are glycosylated products ivacaftor-17-O-D-glucoside (chromatogram peak a), ivacaftor-17-di-O-D-glucoside (chromatogram peak b), and ivacaftor-17-tri-O-D-glucoside (chromatogram peak c).

FIG. 3 is a chart showing water solubility of ivacaftor and ivacaftor-17-O-D-glucoside.

FIG. 4 is a multiple sequence alignment of four UGTs (SEQ ID NOs: 1-4) highlighting similar sequence regions important for catalytic function. The PSPG box is italicized and underlined. The acceptor binding residues are bolded. Sequence Similarity is defined by positive BLAST similarity using the BLOSUM62 scoring matrix and existent: 11, extension: 1 gap penalties.

FIG. 5 shows 3D structures of UGTs indicating the sequence regions that are important for substrate and/or donor binding. All substrates are colored with black carbon sticks (oxygen=red, nitrogen=blue, phosphorous=orange). Cartoon proteins are rainbow from N- to C-terminus. Center: A global structural superposition comprised of multiple UGT crystal structures and homology models. As labeled, zoomed-in regions are clockwise from top-right: I63-G70, C127-T129, V287-Q327, D106-I114. All numbering follows the sequence of SEQ ID NO: 2 with relevant amino acids shown as sticks.

### DETAILED DESCRIPTION

A description of example embodiments follows.

### Cystic Fibrosis

Cystic fibrosis (CF) is caused by mutations in the cystic fibrosis transmembrane conductance regulator (CFTR) gene that result in mislocalization, reduced expression, or reduced activity of the CFTR protein. Expressed on the surface of various absorptive and secretory epithelial cells, CFTR is a cyclic AMP-dependent chloride channel that plays a key role in regulating cellular anion concentrations along with the activity of other ion transporters and proteins (Rafeeq and Murad 2017; Rowe, Miller, and Sorscher 2005). CFTR channel activity and localization are regulated by ATP binding and hydrolysis and by phosphorylation.

Patients diagnosed with CF exhibit imbalances in cellular ion and fluid transport. This leads to a thickening and accumulation of the mucus lining the lungs, intestines, pancreatic ducts, liver, and other organs. In the lungs, the thickened mucus traps pathogenic microbes, leading to an increase in infections and inflammation. This may eventually lead to respiratory failure, the main cause of death in patients with CF (Cystic Fibrosis Foundation 2018). Blockage of the pancreatic ducts prevents the release of digestive enzymes, which leads to poor absorption of nutrients, vitamin deficiencies, malnutrition, and poor growth and development. Other complications that can arise as a result of CF also include liver disease, infertility (especially in men), osteoporosis, electrolyte imbalance, and dehydration (Rafeeq and Murad 2017).

More than 1,700 different CFTR mutations have been described (Cystic Fibrosis Foundation 2018). CFTR mutations are divided into five classes based on the molecular mechanism underlying the reduction or loss of CFTR activity. These mechanisms include failure to express normal levels of CFTR protein due to premature mRNA termination, errors in splicing and promoter regulation, or increased protein turnover (class I, V, and VI respectively); aberrant localization due to improper post-translational modifications (class II); and reduced or abnormal channel activity due to defects in regulation by ATP binding and hydrolysis or due to reduced channel conductance or gating (class III and IV respectively). Class I-III CFTR mutations generally lead to more severe forms of CF. Deletion of a phenylalanine at position 508 (F508del), a class II mutation, is the most common mutation found in CF patients (approximately 90%) (“About Cystic Fibrosis” n.d.). The prevalence of all other CFTR variants is less than 5% (Cystic Fibrosis Foundation 2018).

Diseases that are due to defects in CFTR but do not meet the diagnostic criteria for CF are called CFTR-related diseases. These diseases include late-onset pulmonary disease, congenital bilateral absence of the vas deferens, acute, chronic, or recurrent pancreatitis, sinusitis, allergic bronchopulmonary aspergillosis, smoking-related respiratory diseases, and asthma (Noone and Knowles 2001; Solomon et al. 2016; Flores et al. 2016; Bombieri et al. 2011; Sloane et al. 2012). Furthermore, other diseases that may not be directly caused by defects in CFTR but could benefit from CFTR modulation include any diseases associated with issues in fluid or ion movement and thickened mucus. Examples of these diseases include diarrhea, constipation, asthma, chronic bronchitis, dry eye disease, Sjögren's disease, and chronic obstructive pulmonary disease (Cil et al. 2016; Frossard et al. 2007; Levin and Verkman 2005).

### Ivacaftor

Ivacaftor is a compound represented by the following structural formula:

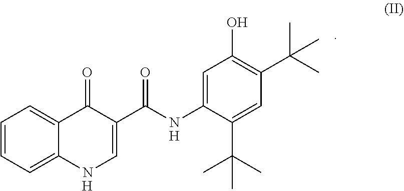

Ivacaftor, also known as VX-770, is a first-in-class drug developed by Vertex Pharmaceuticals that is FDA-approved to treat CF. Ivacaftor directly binds to and modulates the activity of CFTR (Van Goor et al. 2009; Hadida-Ruah et al. 2009). While the exact mechanism of action is still unknown, several studies have shown that ivacaftor promotes CFTR channel opening by a nonconventional ATP-independent mechanism and that ivacaftor likely binds CFTR at an allosteric site (Eckford et al. 2012).

Ivacaftor has been approved to treat CF as a single therapeutic or in combination therapies with other drugs that correct CFTR expression or trafficking issues. Four of these therapies are currently available to CF patients. Ivacaftor (under the trade name KALYDECO®) has been approved to treat CF in patients >6 months of age with one of 38 mutations in the CFTR gene that still produce CFTR protein and that have been shown to respond to ivacaftor in clinical trials or in in vitro assays (“Vertex Pharmaceuticals. KALYDECO (ivacaftor) [package Insert]” 2019). ORKAMBI® (ivacaftor and lumacaftor combination therapy) (“Vertex Pharmaceuticals. ORKAMBI (ivacaftor; Lumacaftor) [package Insert]” 2018) is approved for the treatment of CF in patients aged 2 years and older who are homozygous for the F508del mutation in the CFTR gene. SYMDECO® (ivacaftor and tezacaftor combination therapy) (“Vertex Pharmaceuticals. SYMDECO (ivacaftor; Tezacaftor) [package Insert]” 2019) is approved for the treatment of CF in patients aged 6 years and older who are either homozygous for the F508del mutation in the CFTR gene or who possess at least one copy of one of 25 mutations in the CFTR gene that have been shown to respond to SYMDECO. TRIKAFTA® (ivacaftor, tezacaftor, and elexacaftor combination therapy) (“Vertex Pharmaceuticals. TRIKAFTA (ivacaftor; tezacaftor; elexacaftor) [package Insert]” 2019) is approved for the treatment of CF in patients aged 12 years and older who have at least one copy of the F508del mutation in the CFTR gene.

A meta-analysis of results from clinical trials between January 2005 and March 2018 studying ivacaftor, lumacaftor, tezacaftor, and associated combination therapies (Habib et al. 2019) found that the largest improvement in CF symptoms came from ivacaftor treatment of CF patients with the G55D mutation. Although the effect is much smaller than ivacaftor treatment of G55D CF patients, significant improvement was also seen with combination tezacaftor-ivacaftor or lumacaftor-ivacaftor drug treatment in F508del homozygous patients and for ivacaftor treatment of CF patients with the R117H mutation. No significant improvement in CF symptoms was seen in F508del homozygous patients with ivacaftor, lumacaftor, or tezacaftor single therapeutic treatments, or in F508del heterozygous patients with lumacaftor or lumacaftor-ivacaftor treatments.

While ivacaftor is currently only approved to treat CF, clinical trials are underway to test its efficacy in treating CFTR-related diseases including chronic obstructive bronchial diseases (pulmonary disease, chronic bronchitis, primary ciliary dyskinesia, chronic rhinosinusitis), CF-related diabetes, and CF-related bone disease.

Unfortunately, the low water solubility and the side effects of ivacaftor limit its broader use. Ivacaftor exhibits very low solubility in water (<0.001 mg/ml), which limits the further improvement of its oral bioavailability. The low solubility also complicates the drug formulation process, and may result in unnecessarily high dosages that may increase drug prices and the potential for side effects. Meanwhile, increased respiratory adverse events, especially shortness of breath or dyspnea, is reported with lumacaftor-ivacaftor treatment (Habib et al. 2019). Other side effects include cataracts (especially in pediatric patients), oropharyngeal pain, elevated transaminase levels, and upper respiratory tract infection (Rafeeq and Murad 2017). Furthermore, the effectiveness of ivacaftor is greatly affected by CYP3A inducers and inhibitors (“Vertex Pharmaceuticals. KALYDECO (ivacaftor) [package Insert]” 2019; Jordan, Noah, and Henry 2016). Because CF patients must take a slew of drugs to manage the symptoms of CF, balancing the many possible drug interactions can be difficult. This drug interaction is a particularly important consideration for the lumacaftor-ivacaftor combination therapy. Lumacaftor acts as a CYP3A inducer, necessitating the use of higher doses of both drugs (Rafeeq and Murad 2017). Two patents address the metabolic stability issues of ivacaftor by describing the development of either deuterated or silicon-containing analogs of ivacaftor (WO/2012/158885 A1, Morgan 2012; WO/2017/177124 A1, Munoz, Parks, and Bastos 2017), but there is still a need to address these limitations of ivacaftor treatment in a way that allows ivacaftor to have the maximum effectiveness for the largest number of patients while keeping costs reasonable. Finally, ivacaftor derivatives with altered PK/PD profiles may find useful applications in the treatment of other CFTR-related diseases.

### Glycosylation

A potential strategy for improving or modulating the efficacy, safety, and/or PK/PD profile of a small molecule-based therapeutic such as ivacaftor is modification by glycosylation. The small molecule, or aglycone, is modified by the addition of one or more sugar groups or chains of two or more sugar groups (called oligosaccharides) to nucleophilic centers of the aglycone. These sugar groups can be naturally occurring sugars such as glucose, fructose, rhamnose, mannose, galactose, fucose, xylose, arabinose, glucuronic acid, or N-acetylglucosamine, or they can be synthetically synthesized sugars (e.g., 6-Br-D-glucose, 2-deoxy-D-glucose, 5-thio-D-glucose). These sugars can be attached to the small molecule or to other sugar groups by either an alpha or beta glycosidic bond.

In general, glycosylation of a small molecule can lead to increased aqueous solubility, altered interactions with proteins and membranes, altered absorption and excretion, changes in metabolic stability, and other changes in PK/PD characteristics (Gantt, Peltier-Pain, and Thorson 2011; Kien 2008; De Bruyn et al. 2015).

Glycosylation can enhance or block the transport of a glycoside into specific tissues or organs. Glycosylation can enhance uptake through interaction between the glycoside moiety and lectins or glucose transporters on the cell surface.

In some cases, glycosylation alters the pharmacological activity of the drug, either by enhancing or decreasing potency or even by changing the mechanism of action (Kien 2008; Gantt, Peltier-Pain, and Thorson 2011; De Bruyn et al. 2015).

The identity of the sugar and the stereochemistry of the glycosidic bond can also affect the pharmacological activity or PK/PD profile of a glycoside.

Glycosylation is also a potential strategy for developing prodrugs and compounds for targeted drug delivery to specific tissues. Glycosidases are enzymes that catalyze the hydrolysis of glycosidic bonds and that are specifically expressed in different tissues and organs including blood plasma, the colon, the intestines, and the gut microflora. Glycosidases exhibit substrate specificity towards different glycosidic bond stereochemistry or towards different monosaccharides. A glycosylated drug could function as a prodrug or as a targeted drug if it is preferentially cleaved by a tissue-specific glycosidase. This has been demonstrated by Zipp et al: the alpha-glycosidic bonds in cannabinoid glycosides have been shown to be preferentially cleaved by glycosidases present in the large intestine of mice and not by other chemical or enzymatic processes that may be present in the small intestine, stomach, blood plasma, or brain (Zipp, Hardman, and Brooke 2018; Hardman, Brooke, and Zipp 2017).

In summary, glycosylation of a small molecule may improve aqueous solubility, but may also alter interactions with proteins and membranes, pharmacological activity, and/or PK/PD characteristics in ways that are unexpected.

### Glycosyltransferases

Traditional methods for glycosylating small molecules are non-selective, and it is particularly difficult to control the stereo- and regiospecificity of glycosylation (Zhu and Schmidt 2009; Gu et al. 2014). There is often more than one position on the aglycone that will react with the reagent used to make the desired modification. This makes it necessary to chemically ‘block’ or render temporarily unreactive, the other positions on the molecule in order to selectively modify the desired position. A typical modification will require multiple protection and de-protection steps using the standard methods of synthetic organic chemistry.

Glycosyltransferases (GTs) are a class of enzyme with the potential to act as the catalyst for the generation of novel glycosylated therapeutic small molecules. GTs catalyze the transfer of a sugar from an activated sugar donor molecule to an acceptor molecule (Lairson et al. 2008). They are a large and well-characterized family found in viruses, archaea, bacteria, and eukaryotes. Greater than 600,000 GTs categorized into approximately 110 families are described in the Carbohydrate-active Enzymes Directory (www.cazy.org), and greater than 150 GT structures are reported (www.rcsb.org) (Lombard et al. 2014; Berman 2000). The majority of GTs utilize nucleotide-activated sugar donors and are referred to as Leloir GTs, although lipid phosphate and phosphate-activated sugar donors are also used (Breton, Fournel-Gigleux, and Palcic 2012; Lairson et al. 2008). GT acceptors include proteins, lipids, oligosaccharides, and small molecules.

GTs offer several advantages as a potential tool in a general small molecule glycosylation platform (De Bruyn et al. 2015; Gantt, Peltier-Pain, and Thorson 2011; Yonekura-Sakakibara and Hanada 2011; Schmid et al. 2016). GTs are often characterized by very high conversion efficiencies (up to 100%). As a result, lower concentrations of potentially expensive or difficult to synthesize substrates are required for GT-catalyzed reactions. GTs are able to glycosylate a wide variety of acceptor structures, with many GTs exhibiting promiscuity towards the sugar donor and acceptor. Furthermore, GTs can catalyze the formation of O-, N-, S-, and even C-glycosides. As a result of these characteristics, GTs are generally amenable to both in vitro and in vivo bioengineering efforts.

### Uridine Diphosphate GTs (UGTs)

Uridine diphosphate GTs (UGTs) utilize uridine diphosphate (UDP) sugar donors, and form the largest group of Leloir GTs in plants (Yonekura-Sakakibara and Hanada 2011). Recently, the identification and characterization of new UGTs, especially in plants and bacteria, has exploded as part of an increased interest in characterizing natural product biosynthetic pathways. This method is described by Torens-Spence et al. (Torrens-Spence et al. 2018). In this paper, 33 UGT enzyme-encoding genes were cloned from a Golden root plant, expressed in yeast, and screened for regiospecific activity in modifying tyrosol to produce salidroside or icariside D2, which are tyrosol metabolites in the plant's native salidroside biosynthetic pathway. Another group identified naturally occurring enzymes having promiscuous N- and O-glycosyltransferase activity by mining the expressed genes of Carthamus tinctorius. K. Xie et al. (Xie et al. 2017) describes the identification of a promiscuous glycosyltransferase (UGT71E5) from C. tinctorius which contains N-glycosylase activity towards multiple diverse nitrogen-heterocyclic aromatic compounds. Zhang et al. (Zhang et al. 2019) describes the identification of three new UGTs (UGT 84A33, UGT 71AE1 and UGT 90A14) from C. tinctorius having promiscuous O-glycosyltransferase activity against benzylisoquinoline alkaloids and their use in making glycosylated derivatives. With the continuing technological improvements and decreasing costs of genome and transcriptome sequencing and analysis, it is becoming easier to identify and characterize naturally occurring GTs for the development of novel small molecule diversity generating platforms.

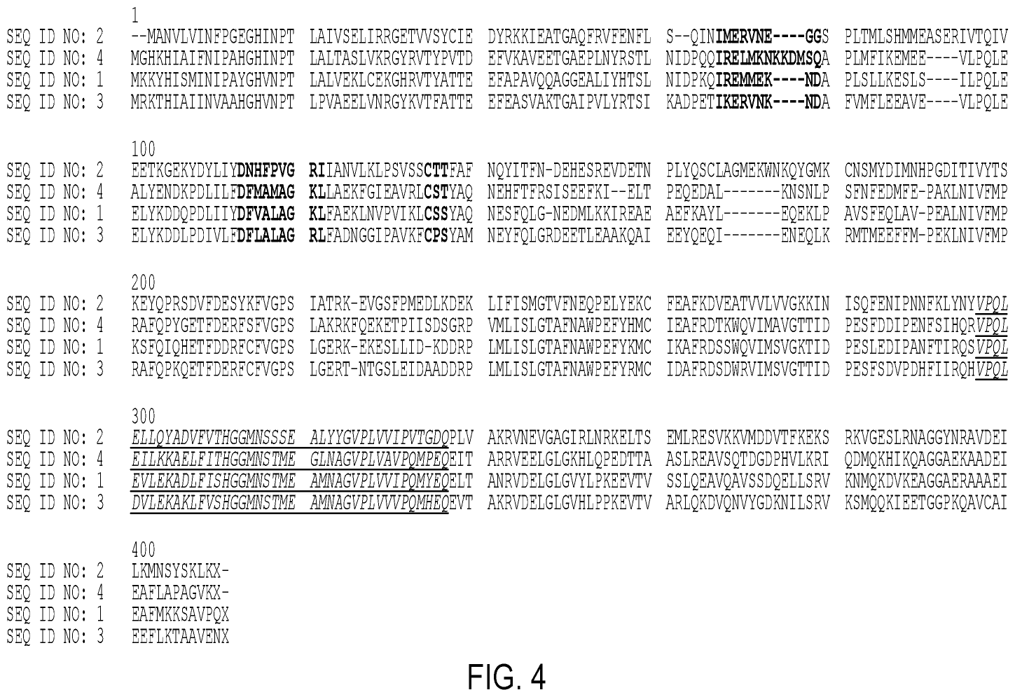

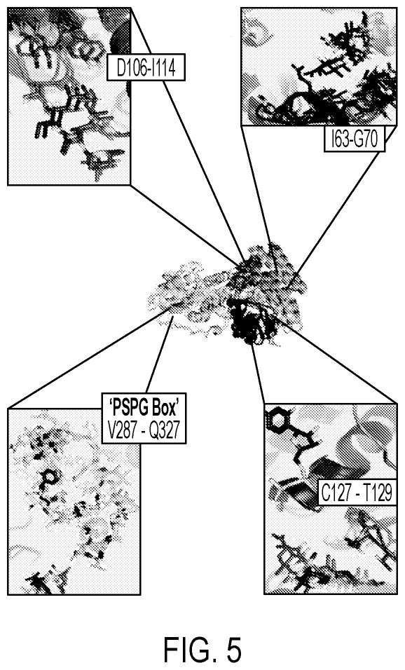

As described herein, four regions within UGT sequences are identified as important for activity. The sequences of all four regions in SEQ ID NO: 1-4 are unique in comparison to other UGTs but highly similar among themselves (FIG. 4). This indicates a strong correlation between the sequences within the four regions and those enzymes' unique activity toward ivacaftor. Three acceptor binding sites are shown in crystal structures (or homology models) as poised to interact with sugar acceptor molecules. The “PSPG Box” region is involved in both UGT donor and acceptor substrate affinity and is likely a major part of specific activity (FIG. 5) (Bairoch 1991; Hughes and Hughes 1994; Yamazaki, Gong et al. 1999; Hans, Brandt et al. 2004; Shao, He et al. 2005; He, Wang et al. 2006; Offen, Martinez-Fleites et al. 2006).

TABLE 1
UGT Enzyme Regions Important for Activity

| | | | Sequence |
|---|---|---|---|
| Enzyme | Region | Function | Similarity* |
| SEQ ID NO: 1 | I67 - D75 | Acceptor Substrate Binding | 90% |
| (uridine diphosphate glycosyltransferase | D106 - L114 | Acceptor Substrate Binding | 90% |
| (UGT) from Bacillus subtilis) | C127- S129 | Acceptor Substrate Binding | 90% |
| | V278 - Q318 | “PSPG Box” - Donor/Acceptor Binding | 80% |
| SEQ ID NO: 2 | I63- G70 | Acceptor Substrate Binding | 90% |
| (uridine diphosphate glycosyltransferase | D106 - I114 | Acceptor Substrate Binding | 90% |
| (UGT) from Bacillus cereus) | C127 - T129 | Acceptor Substrate Binding | 90% |
| | V287 - Q327 | “PSPG Box” - Donor/Acceptor Binding | 80% |
| SEQ ID NO: 3 | I67 - D75 | Acceptor Substrate Binding | 90% |
| (uridine diphosphate glycosyltransferase | D106 - L114 | Acceptor Substrate Binding | 90% |
| (UGT) from Bacillus methylotrophicus) | C127 - S129 | Acceptor Substrate Binding | 90% |
| | V280 - Q320 | “PSPG Box” - Donor/Acceptor Binding | 80% |
| SEQ ID NO: 4 | I67 - Q79 | Acceptor Substrate Binding | 90% |
| (uridine diphosphate glycosyltransferase | D110 - L118 | Acceptor Substrate Binding | 90% |
| (UGT) from Bacillus licheniformis) | C131 - T133 | Acceptor Substrate Binding | 90% |
| | V283 - Q323 | “PSPG Box” - Donor/Acceptor Binding | 80% |

*Sequence Similarity is defined by positive BLAST similarity using the BLOSUM62 scoring matrix and existent: 11, extension: 1 gap penalties (Altschul et al. 1990; Henikoff et al. 1992). A commonly used tool for determining percent sequence identity is Protein Basic Local Alignment Search Tool (BLASTp) available through National Center for Biotechnology Information, National Library of Medicine, of the United States National Institutes of Health.

In some embodiments, the UGT includes an amino acid sequence that is at least 80% (85%, 90%, 91%, 92%, 93%, 94%, 95% 96%, 97%, 98%, 99%, or 100%) similar to SEQ ID NO: 1. In some embodiments, the UGT includes an amino acid sequence that is at least 80% (85%, 90%, 91%, 92%, 93%, 94%, 95% 96%, 97%, 98%, 99%, or 100%) identical to SEQ ID NO: 1.

In some embodiments, the UGT includes an amino acid sequence that is at least 80% (85%, 90%, 91%, 92%, 93%, 94%, 95%, 96%, 97%, 98%, 99%, or 100%) similar to a region from V278 to Q318 of SEQ ID NO: 1. In some embodiments, the UGT includes an amino acid sequence that is at least 80% (85%, 90%, 91%, 92%, 93%, 94%, 95%, 96%, 97%, 98%, 99%, or 100%) identical to a region from V278 to Q318 of SEQ ID NO: 1.

In some embodiments, the UGT includes an amino acid sequence that is: at least 90% (91%, 92%, 93%, 94%, 95%, 96%, 97%, 98%, 99%, or 100%) similar to a region from 167 to D75 of SEQ ID NO: 1; at least 90% (91%, 92%, 93%, 94%, 95%, 96%, 97%, 98%, 99%, or 100%) similar to a region from D106 to L114 of SEQ ID NO: 1; at least 90% (91%, 92%, 93%, 94%, 95%, 96%, 97%, 98%, 99%, or 100%) similar to a region from C127 to S129 of SEQ ID NO: 1; and at least 80% (85%, 90%, 91%, 92%, 93%, 94%, 95%, 96%, 97%, 98%, 99%, or 100%) similar to a region from V278 to Q318 of SEQ ID NO: 1.

In some embodiments, the UGT includes an amino acid sequence that is: at least 90% (91%, 92%, 93%, 94%, 95%, 96%, 97%, 98%, 99%, or 100%) identical to a region from 167 to D75 of SEQ ID NO: 1; at least 90% (91%, 92%, 93%, 94%, 95%, 96%, 97%, 98%, 99%, or 100%) identical to a region from D106 to L114 of SEQ ID NO: 1; at least 90% (91%, 92%, 93%, 94%, 95%, 96%, 97%, 98%, 99%, or 100%) identical to a region from C127 to S129 of SEQ ID NO: 1; and at least 80% (85%, 90%, 91%, 92%, 93%, 94%, 95%, 96%, 97%, 98%, 99%, or 100%) identical to a region from V278 to Q318 of SEQ ID NO: 1.

In some embodiments, the UGT includes an amino acid sequence that is at least 80% (85%, 90%, 91%, 92%, 93%, 94%, 95% 96%, 97%, 98%, 99%, or 100%) similar to SEQ ID NO: 2. In some embodiments, the UGT includes an amino acid sequence that is at least 80% (85%, 90%, 91%, 92%, 93%, 94%, 95% 96%, 97%, 98%, 99%, or 100%) identical to SEQ ID NO: 2.

In some embodiments, the UGT includes an amino acid sequence that is at least 80% (85%, 90%, 91%, 92%, 93%, 94%, 95%, 96%, 97%, 98%, 99%, or 100%) similar to a region from V287 to Q327 of SEQ ID NO: 2. In some embodiments, the UGT includes an amino acid sequence that is at least 80% (85%, 90%, 91%, 92%, 93%, 94%, 95%, 96%, 97%, 98%, 99%, or 100%) identical to a region from V287 to Q327 of SEQ ID NO: 2.

In some embodiments, the UGT includes an amino acid sequence that is: at least 90% (91%, 92%, 93%, 94%, 95%, 96%, 97%, 98%, 99%, or 100%) similar to a region from 163 to G70 of SEQ ID NO: 2; at least 90% (91%, 92%, 93%, 94%, 95%, 96%, 97%, 98%, 99%, or 100%) similar to a region from D106 to 1114 of SEQ ID NO: 2; at least 90% (91%, 92%, 93%, 94%, 95%, 96%, 97%, 98%, 99%, or 100%) similar to a region from C127 to T129 of SEQ ID NO: 2; and at least 80% (85%, 90%, 91%, 92%, 93%, 94%, 95%, 96%, 97%, 98%, 99%, or 100%) similar to a region from V287 to Q327 of SEQ ID NO: 2.

In some embodiments, the UGT includes an amino acid sequence that is: at least 90% (91%, 92%, 93%, 94%, 95%, 96%, 97%, 98%, 99%, or 100%) identical to a region from 163 to G70 of SEQ ID NO: 2; at least 90% (91%, 92%, 93%, 94%, 95%, 96%, 97%, 98%, 99%, or 100%) identical to a region from D106 to 1114 of SEQ ID NO: 2; at least 90% (91%, 92%, 93%, 94%, 95%, 96%, 97%, 98%, 99%, or 100%) identical to a region from C127 to T129 of SEQ ID NO: 2; and at least 80% (85%, 90%, 91%, 92%, 93%, 94%, 95%, 96%, 97%, 98%, 99%, or 100%) identical to a region from V287 to Q327 of SEQ ID NO: 2.

In some embodiments, the UGT includes an amino acid sequence that is at least 80% (85%, 90%, 91%, 92%, 93%, 94%, 95% 96%, 97%, 98%, 99%, or 100%) similar to SEQ ID NO: 3. In some embodiments, the UGT includes an amino acid sequence that is at least 80% (85%, 90%, 91%, 92%, 93%, 94%, 95% 96%, 97%, 98%, 99%, or 100%) identical to SEQ ID NO: 3.

In some embodiments, the UGT includes an amino acid sequence that is at least 80% (85%, 90%, 91%, 92%, 93%, 94%, 95%, 96%, 97%, 98%, 99%, or 100%) similar to a region from V280 to Q320 of SEQ ID NO: 3. In some embodiments, the UGT includes an amino acid sequence that is at least 80% (85%, 90%, 91%, 92%, 93%, 94%, 95%, 96%, 97%, 98%, 99%, or 100%) identical to a region from V280 to Q320 of SEQ ID NO: 3.

In some embodiments, the UGT includes an amino acid sequence that is: at least 90% (91%, 92%, 93%, 94%, 95%, 96%, 97%, 98%, 99%, or 100%) similar to a region from 167 to D75 of SEQ ID NO: 3; at least 90% (91%, 92%, 93%, 94%, 95%, 96%, 97%, 98%, 99%, or 100%) similar to a region from D106 to L114 of SEQ ID NO: 3; at least 90% (91%, 92%, 93%, 94%, 95%, 96%, 97%, 98%, 99%, or 100%) similar to a region from C127 to S129 of SEQ ID NO: 3; and at least 80% (85%, 90%, 91%, 92%, 93%, 94%, 95%, 96%, 97%, 98%, 99%, or 100%) similar to a region from V280 to Q320 of SEQ ID NO: 3.

In some embodiments, the UGT includes an amino acid sequence that is: at least 90% (91%, 92%, 93%, 94%, 95%, 96%, 97%, 98%, 99%, or 100%) identical to a region from 167 to D75 of SEQ ID NO: 3; at least 90% (91%, 92%, 93%, 94%, 95%, 96%, 97%, 98%, 99%, or 100%) identical to a region from D106 to L114 of SEQ ID NO: 3; at least 90% (91%, 92%, 93%, 94%, 95%, 96%, 97%, 98%, 99%, or 100%) identical to a region from C127 to S129 of SEQ ID NO: 3; and at least 80% (85%, 90%, 91%, 92%, 93%, 94%, 95%, 96%, 97%, 98%, 99%, or 100%) identical to a region from V280 to Q320 of SEQ ID NO: 3.

In some embodiments, the UGT includes an amino acid sequence that is at least 80% (85%, 90%, 91%, 92%, 93%, 94%, 95% 96%, 97%, 98%, 99%, or 100%) similar to SEQ ID NO: 4. In some embodiments, the UGT includes an amino acid sequence that is at least 80% (85%, 90%, 91%, 92%, 93%, 94%, 95% 96%, 97%, 98%, 99%, or 100%) identical to SEQ ID NO: 4.

In some embodiments, the UGT includes an amino acid sequence that is at least 80% (85%, 90%, 91%, 92%, 93%, 94%, 95%, 96%, 97%, 98%, 99%, or 100%) similar to a region from V283 to Q323 of SEQ ID NO: 4. In some embodiments, the UGT includes an amino acid sequence that is at least 80% (85%, 90%, 91%, 92%, 93%, 94%, 95%, 96%, 97%, 98%, 99%, or 100%) identical to a region from V283 to Q323 of SEQ ID NO: 4.

In some embodiments, the UGT includes an amino acid sequence that is: at least 90% (91%, 92%, 93%, 94%, 95%, 96%, 97%, 98%, 99%, or 100%) similar to a region from 167 to Q79 of SEQ ID NO: 4; at least 90% (91%, 92%, 93%, 94%, 95%, 96%, 97%, 98%, 99%, or 100%) similar to a region from D110 to L118 of SEQ ID NO: 4; at least 90% (91%, 92%, 93%, 94%, 95%, 96%, 97%, 98%, 99%, or 100%) similar to a region from C131 to T133 of SEQ ID NO: 4; and at least 80% (85%, 90%, 91%, 92%, 93%, 94%, 95%, 96%, 97%, 98%, 99%, or 100%) similar to a region from V283 to Q323 of SEQ ID NO: 4.

In some embodiments, the UGT includes an amino acid sequence that is: at least 90% (91%, 92%, 93%, 94%, 95%, 96%, 97%, 98%, 99%, or 100%) identical to a region from 167 to Q79 of SEQ ID NO: 4; at least 90% (91%, 92%, 93%, 94%, 95%, 96%, 97%, 98%, 99%, or 100%) identical to a region from D110 to L118 of SEQ ID NO: 4; at least 90% (91%, 92%, 93%, 94%, 95%, 96%, 97%, 98%, 99%, or 100%) identical to a region from C131 to T133 of SEQ ID NO: 4; and at least 80% (85%, 90%, 91%, 92%, 93%, 94%, 95%, 96%, 97%, 98%, 99%, or 100%) identical to a region from V283 to Q323 of SEQ ID NO: 4.

### Monosaccharides, Disaccharides, Trisaccharides, and Oligosaccharides

Glycosyltransferases can catalyze the addition of many different monosaccharides to ivacaftor. In general, suitable monosaccharides include, but are not limited to, open and closed chain monosaccharides. The monosaccharides can be in the L- or D-configuration. Typically, the monosaccharides have 5, 6, or 7 carbons (a pentose monosaccharide, hexose monosaccharide, or heptose monosaccharide, respectively).

Suitable monosaccharides include allose, apiose, arabinose, fructose, fucitol, fucose, galactose, glucose, glucuronic acid, mannose, N-acetylglucosamine, N-acetylgalactosamine, rhamnose, and xylose. Other suitable monosaccharides include glucosamine, galactosamine, mannosamine, 5-thio-D-glucose, nojirimycin, deoxynoirimycin, 1,5-anhydro-D-sorbitol, 2,5-anhydro-D-mannitol, 2-deoxy-D-galactose, 2-deoxy-D-glucose, 3-deoxy-D-glucose, arabinitol, galactitol, glucitol, iditol, lyxose, mannitol, L-rhamnitol, 2-deoxy-D-ribose, ribose, ribitol, ribulose, xylulose, altrose, gulose, idose, levulose, psicose, sorbose, tagatose, talose, galactal, glucal, fucal, rhamnal, arabinal, xylal, 3,4-di-O-acetyl-L-fucal, 3,4-di-O-acetyl-L-rhamnal, 3,4-di-O-acetyl-D-arabinal, 3,4-di-O-acetyl-D-xylal, valienamine, validamine, valiolamine, valienol, valienone, galacturonic acid, mannuronic acid, N-acetylneuraminic acid, N-acetylmuramic acid, gluconic acid D-lactone, galactonic acid gamma-lactone, galactonic acid delta-lactone, mannonic acid gamma-lactone, D-altro-heptulose, D-manno-heptulose, D-glycero-D-manno-heptose, D-glycero-D-gluco-heptose, D-allo-heptulose, D-altro-3-heptulose, D-glycero-D-manno-heptitol, and D-glycero-D-altro-heptitol.

Suitable oligosaccharides include, but are not limited to, carbohydrates having from 2 to 10 or more monosaccharides linked together (e.g., 2, 3, 4, 5, 6, 7, 8, 9, or 10 monosaccharides linked together). The constituent monosaccharide unit may be, for example, a pentose monosaccharide, a hexose monosaccharide, or a pseudosugar (including a pseudoamino sugar). Oligosaccharides do not include bicyclic groups that are formed by fusing a monosaccharide to a benzene ring, a cyclohexane ring, or a heterocyclic ring. Pseudosugars that may be used in the invention are members of the class of compounds wherein the ring oxygen atom of the cyclic monosaccharide is replaced by a methylene group. Pseudosugars are also known as “carba-sugars.”

The glycosyltransferases can catalyze addition of a monosaccharide to ivacaftor, and the bond between the monosaccharide and ivacaftor can be either an alpha or beta glycosidic bond. Disaccharides, trisaccharides, and oligosaccharides are formed by serial enzymatic additions of two or more monosaccharides to ivacaftor. When more than one monosaccharide is added by serial enzymatic reactions, successive monosaccharides can be bonded to the preceding monosaccharide by either an alpha or beta glycosidic bond.

### Methods of Making Ivacaftor Glycosides

Ivacaftor glycosides can be made from ivacaftor by an enzymatically catalyzed reaction. A reaction mixture is provided that includes ivacaftor, a uridine diphosphate glycosyltransferase, and a uridine diphosphate-monosaccharide. After a period of time (e.g., from 1 to 72 hours), ivacaftor is converted to a monosaccharide, disaccharide, trisaccharide, or oligosaccharide of ivacaftor. The monosaccharide, disaccharide, trisaccharide, or oligosaccharide of ivacaftor that is formed corresponds to the uridine diphosphate-monosaccharide that is included in the reaction mixture.

In some embodiments, the UGT enzyme and recombinant UGT-expressing cell lysate (e.g., yeast cell lysate) are placed in a reaction vessel. To form the lysate, UGT-expressing cells (e.g., UGT-expressing yeast cells) are lysed and the insoluble part is discarded by centrifugation so that the lysate is cell-free. In other embodiments, the cell-free lysate is not required. For example, in some embodiments, recombinant UGTs can be used. In other embodiments, purified UGTs can be used.

### Ivacaftor Glycosides

Ivacaftor glycosides are compounds represented by the following structural formula:

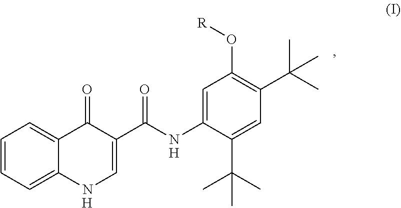

R is a monosaccharide, disaccharide, trisaccharide, or an oligosaccharide comprising 4 to 10 monosaccharides (e.g. 4, 5, 6, 7, 8, 9, or 10 monosaccharides). In some instances, the compound is a pharmaceutically acceptable salt of Compound (I).

In one embodiment, R is glucose, which can be D-glucose or L-glucose. D-glucose is represented by the following structural formula:

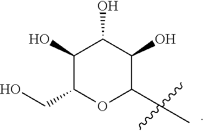 [SMILES: *C1O[C@H](CO)[C@@H](O)[C@H](O)[C@H]1O]

In one embodiment, R is galactose, which can be D-galactose or L-galactose. D-galactose is represented by the following structural formula:

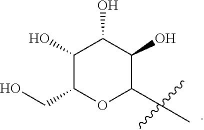 [SMILES: *C1O[C@H](CO)[C@H](O)[C@H](O)[C@H]1O]

In one embodiment, R is xylose, which can be D-xylose or L-xylose. Xylose can form six- and five-membered rings. A five-membered ring of D-xylose is represented by the following structural formula:

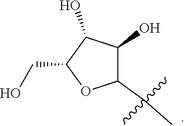 [SMILES: *C1O[C@H](CO)[C@H](O)[C@H]1O]

In one embodiment, R is N-acetylglucosamine, which can be D-N-acetylglucosamine or L-N-acetylglucosamine. D-N-acetylglucosamine is represented by the following structural formula:

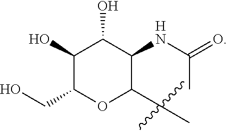

The bond between the monosaccharide (e.g., glucose) and ivacaftor can be an alpha or beta glycosidic bond. The bond between monosaccharides of a disaccharide can be either an alpha or beta glycosidic bond. The bond between monosaccharides of a trisaccharide can be either an alpha or beta glycosidic bond. The bond between monosaccharides of an oligosaccharide can be either an alpha or beta glycosidic bond. The glycosidic bond between monosaccharides of a disaccharide or trisaccharide and between monosaccharides of an oligosaccharide can be formed between any of the hydroxyl groups from each monosaccharide. In other words, the bond between monosaccharides can be, e.g., 1→2, 1→3, 1→4, or 1→6.

In some embodiments, R is a disaccharide.

In one embodiment, R is a disaccharide consisting of two molecules of glucose, and the compound is ivacaftor-17-di-O-D-glucoside. A disaccharide consisting of two monomers of glucose, where the two monomers are bonded by a 1→2 glycosidic bond, has the following structural formula:

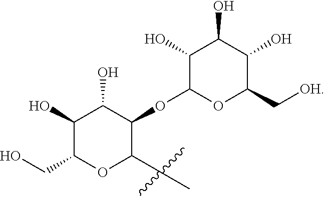 [SMILES: *C1O[C@H](CO)[C@@H](O)[C@H](O)[C@H]1OC1O[C@H](CO)[C@@H](O)[C@H](O)[C@H]1O]

In one embodiment, R is a disaccharide consisting of two molecules of galactose, and the compound is ivacaftor-17-di-O-D-galactoside. A disaccharide consisting of two monomers of galactose, where the two monomers are bonded by a 1->2 glycosidic bond, has the following structural formula:

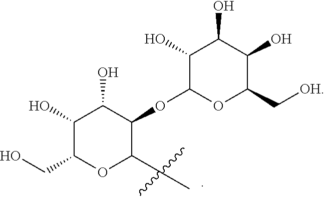 [SMILES: *C1O[C@H](CO)[C@H](O)[C@H](O)[C@H]1OC1O[C@H](CO)[C@H](O)[C@H](O)[C@H]1O]

In one particular embodiment, R is a disaccharide consisting of two molecules of xylose, and the compound is ivacaftor-17-di-O-D-xyloside. A disaccharide consisting of two monomers of xylose, where the two monomers are bonded by a 1→2 glycosidic bond, has the following structural formula:

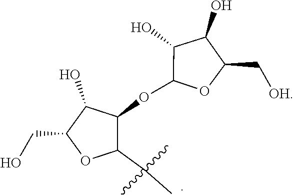 [SMILES: *C1O[C@H](CO)[C@H](O)[C@H]1OC1O[C@H](CO)[C@H](O)[C@H]1O]

In some embodiments, the disaccharide includes two different monosaccharides. In some embodiments, the trisaccharide or oligosaccharide includes two or more different monosaccharides. One example is ivacaftor-17-O-xylose-glucoside.

In some embodiments, R is a trisaccharide.

In one embodiment, R is a trisaccharide consisting of three molecules of glucose, and the compound is ivacaftor-17-tri-O-D-glucose. The three monomers are bonded by a 1→2 or 1→4 glycosidic bond. A trisaccharide consisting of three monomers of glucose, where two monomers are bonded to a third monomer by a 1→2 glycosidic bond and by a 1→4 glycosidic bond, wherein the third monomer is bonded to ivacaftor, has the following structural formula:

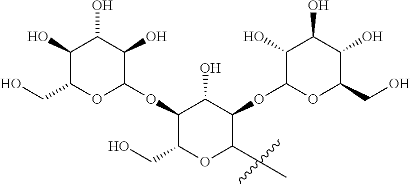 [SMILES: *C1O[C@H](CO)[C@@H](OC2O[C@H](CO)[C@@H](O)[C@H](O)[C@H]2O)[C@H](O)[C@H]1OC1O[C@H](CO)[C@@H](O)[C@H](O)[C@H]1O]

### Methods of Treating Diseases

The ivacaftor glycosides described herein can be used in methods of treating diseases. The ivacaftor glycoside is administered to a patient in need thereof.

Diseases that can be treated by administering the ivacaftor glycosides disclosed herein include, but are not limited to, cystic fibrosis, late-onset pulmonary disease, congenital bilateral absence of the vas deferens, acute, chronic, or recurrent pancreatitis, sinusitis, allergic bronchopulmonary aspergillosis, smoking-related respiratory diseases, and asthma (Noone and Knowles 2001; Solomon et al. 2016; Flores et al. 2016; Bombieri et al. 2011; Sloane et al. 2012). Furthermore, other diseases that may not be directly caused by defects in CFTR but could benefit from CFTR modulation include diseases associated with issues in fluid or ion movement and thickened mucus. Examples of these diseases include diarrhea, constipation, asthma, chronic bronchitis, dry eye disease, Sjögren's disease, and chronic obstructive pulmonary disease (Cil et al. 2016; Frossard et al. 2007; Levin and Verkman 2005). Other diseases include chronic obstructive bronchial diseases (pulmonary disease, chronic bronchitis, primary ciliary dyskinesia, chronic rhinosinusitis), CF-related diabetes, and CF-related bone disease.

Typically, patients in need thereof have at least one mutation that is responsive to ivacaftor. A mutation is responsive to ivacaftor if administration of ivacaftor yields a 10% increase in chloride ion transport compared to baseline in an in vitro assay, or improvement in clinical parameters in clinical trials (usually Forced Expiratory Volume, or FEV). Common CFTR mutations include: G551D; F508del; E56K; P67L; R74W; D110E; D110H; R117C; R117H; G178R; E193K; L206W; R347H; R352Q; A455E; S549N; S549R; G551S; D579G; 711+3A>G (splice mutation; intron); E831X (splice mutation; X is a stop codon); S945L; S977F; F1052V; K1060T; A1067T; G1069R; R1070Q; R1070W; F1074L; D1152H; G1244E; S1251N; S1255P; D1270N; G1349D; 2789+5G>A (splice mutation; intron); 3272-26A>G (splice mutation; end of intron); 3849+10kbC>T (splice mutation; intron).

Treatment with ivacaftor for F508del is only indicated if F508del is on one CFTR allele and the other allele is one of these mutations: G551D, R117H-5T (splice mutation).

The ivacaftor glycosides can be administered as part of a combination therapy.

One example of a combination therapy is ivacaftor-lumacaftor, which is approved for the treatment of CF in patients age 2 years and older who are homozygous for the F508del mutation in the CFTR gene.

A second example of a combination therapy is ivacaftor-tezacaftor, which is approved for the treatment of CF in patients age 6 years and older who are either homozygous for the F508del mutation in the CFTR gene OR who possess at least one copy of one of 25 mutations in the CFTR gene that have been shown to respond to Symdeko. Mutations shown to respond to Symdeko: E56K; P67L; R74W; D110E; D110H; R117C; E193K; L206W; R347H; R352Q; A455E; D579G; 711+3A>G (splice mutation in intron); E831X (X is a stop codon); S945L; S977F; F1052V; K1060T; A1067T; R1070W; F1074L; D1152H; D1270N; 2789+5G>A (splice mutation in intron); 3272-26A>G (splice mutation at end of intron); 3849+10kbC>T (splice mutation in intron).

A third example of a combination therapy is ivacaftor-tezacaftor-elexacaftor, which is approved for the treatment of CF in patients age 12 years and older who have at least one copy of the F508del mutation in the CFTR gene.

The ivacaftor glycosides described herein can be used in place of, or in addition to, ivacaftor in those combination therapies.

### Pharmaceutical Compositions, Dosing, and Administration

Also provided herein is a pharmaceutical composition, comprising an ivacaftor glycoside disclosed herein, or a pharmaceutically acceptable salt thereof, and optionally a pharmaceutically acceptable carrier. The compositions can be used in the methods described herein, e.g., to supply a compound described herein, or a pharmaceutically acceptable salt thereof.

“Pharmaceutically acceptable salt” refers to those salts which are, within the scope of sound medical judgment, suitable for use in contact with the tissues of mammals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, the relevant teachings of which are incorporated herein by reference in their entirety. Pharmaceutically acceptable salts of the compounds described herein include salts derived from suitable inorganic and organic acids, and suitable inorganic and organic bases.

Examples of pharmaceutically acceptable acid addition salts are salts of an amino group formed with inorganic acids such as hydrochloric acid, hydrobromic acid, phosphoric acid, sulfuric acid and perchloric acid, or with organic acids such as acetic acid, oxalic acid, maleic acid, tartaric acid, citric acid, succinic acid or malonic acid or by using other methods used in the art, such as ion exchange. Other pharmaceutically acceptable acid addition salts include adipate, alginate, ascorbate, aspartate, benzenesulfonate, benzoate, bisulfate, borate, butyrate, camphorate, camphorsulfonate, cinnamate, citrate, cyclopentanepropionate, digluconate, dodecylsulfate, ethanesulfonate, formate, fumarate, glucoheptonate, glycerophosphate, gluconate, glutarate, glycolate, hemisulfate, heptanoate, hexanoate, hydroiodide, hydroxybenzoate, 2-hydroxy-ethanesulfonate, hydroxymaleate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 2-phenoxybenzoate, phenylacetate, 3-phenylpropionate, phosphate, pivalate, propionate, pyruvate, salicylate, stearate, succinate, sulfate, tartrate, thiocyanate, p-toluenesulfonate, undecanoate, valerate salts, and the like.

Either the mono-, di- or tri-acid salts can be formed, and such salts can exist in either a hydrated, solvated or substantially anhydrous form.

Salts derived from appropriate bases include salts derived from inorganic bases, such as alkali metal, alkaline earth metal, and ammonium bases, and salts derived from aliphatic, alicyclic or aromatic organic amines, such as methylamine, trimethylamine and picoline, or N*((C1-C4)alkyl)₄ salts. Representative alkali or alkaline earth metal salts include sodium, lithium, potassium, calcium, magnesium, barium and the like. Further pharmaceutically acceptable salts include, when appropriate, nontoxic ammonium, quaternary ammonium, and amine cations formed using counterions such as halide, hydroxide, carboxyl, sulfate, phosphate, nitrate, lower alkyl sulfonate and aryl sulfonate.

“Pharmaceutically acceptable carrier” refers to a non-toxic carrier or excipient that does not destroy the pharmacological activity of the agent with which it is formulated and is nontoxic when administered in doses sufficient to deliver a therapeutic amount of the agent. Pharmaceutically acceptable carriers that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions provided herein can be orally administered in any orally acceptable dosage form including, but not limited to, capsules, tablets, aqueous suspensions, dispersions and solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions and/or emulsions are required for oral use, the active ingredient can be suspended or dissolved in an oily phase and combined with emulsifying and/or suspending agents. If desired, certain sweetening, flavoring or coloring agents may also be added.

In some embodiments, an oral formulation is formulated for immediate release or sustained/delayed release.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders, such as carboxymethylcellulose, alginates, gelatin, polyvinylpyrrolidinone, sucrose, and acacia, (c) humectants such as glycerol, (d) disintegrating agents such as agar-agar, calcium carbonate, potato or tapioca starch, alginic acid, certain silicates, and sodium carbonate, (e) solution retarding agents such as paraffin, (f) absorption accelerators such as quaternary ammonium salts, (g) wetting agents, such as acetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Liquid dosage forms for oral administration include pharmaceutically acceptable emulsions, microemulsions, solutions, suspensions, syrups, and elixirs. In addition to the ivacaftor glycosides of the present disclosure, the liquid dosage forms may contain inert diluents commonly used in the art, such as water or other solvents, solubilizing agents and emulsifiers, such as ethyl alcohol (ethanol), isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, or mixtures thereof. Besides inert diluents, the oral compositions can also include adjuvants such as wetting agents, emulsifying and suspending agents, sweetening, flavoring, coloring, perfuming, and preservative agents.

Compositions suitable for buccal or sublingual administration include tablets, lozenges and pastilles, wherein the active ingredient is formulated with a carrier such as sugar and acacia, tragacanth, or gelatin and glycerin.

Solid compositions of a similar type may also be employed as fillers in soft and hard-filled gelatin capsules using excipients such as lactose or milk sugar, as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

An ivacaftor glycoside described herein can also be in micro-encapsulated form with one or more excipients, as noted above. In such solid dosage forms, the ivacaftor glycoside can be admixed with at least one inert diluent such as sucrose, lactose or starch. Such dosage forms can also comprise, as is normal practice, additional substances other than inert diluents, e.g., tableting lubricants and other tableting aids such a magnesium stearate and microcrystalline cellulose.

Compositions for oral administration may be designed to protect the active ingredient against degradation as it passes through the alimentary tract, for example, by an outer coating of the formulation on a tablet or capsule.

In another embodiment, an ivacaftor glycoside or pharmaceutically acceptable salt described herein can be provided in an extended (or “delayed” or “sustained”) release composition. This delayed-release composition includes the ivacaftor glycoside or pharmaceutically acceptable salt in combination with a delayed-release component. Such a composition allows targeted release of a provided agent into the lower gastrointestinal tract, for example, into the small intestine, the large intestine, the colon and/or the rectum. In certain embodiments, a delayed-release composition further includes an enteric or pH-dependent coating, such as cellulose acetate phthalates and other phthalates (e.g., polyvinyl acetate phthalate, methacrylates (Eudragits)). Alternatively, the delayed-release composition provides controlled release to the small intestine and/or colon by the provision of pH sensitive methacrylate coatings, pH sensitive polymeric microspheres, or polymers which undergo degradation by hydrolysis. The delayed-release composition can be formulated with hydrophobic or gelling excipients or coatings. Colonic delivery can further be provided by coatings which are digested by bacterial enzymes such as amylose or pectin, by pH dependent polymers, by hydrogel plugs swelling with time (Pulsincap), by time-dependent hydrogel coatings and/or by acrylic acid linked to azoaromatic bonds coatings.

The amount of an ivacaftor glycoside described herein, or a pharmaceutically acceptable salt thereof, that can be combined with the carrier materials to produce a composition in a single dosage form will vary depending upon the host treated, the particular mode of administration and the activity of the agent employed. Preferably, compositions should be formulated so that a dosage of from about 0.01 mg/kg to about 100 mg/kg body weight/day of the ivacaftor glycoside, or pharmaceutically acceptable salt thereof, can be administered to a subject receiving the composition.

The desired dose may conveniently be administered in a single dose or as multiple doses administered at appropriate intervals such that, for example, the agent is administered 2, 3, 4, 5, 6 or more times per day. The daily dose can be divided, especially when relatively large amounts are administered, or as deemed appropriate, into several, for example 2, 3, 4, 5, 6 or more, administrations.

It should also be understood that a specific dosage and treatment regimen for any particular patient will depend upon a variety of factors, including the activity of the specific agent employed, the age, body weight, general health, sex, diet, time of administration, rate of excretion, drug combination, the judgment of the treating physician and the severity of the particular disease being treated. The amount of an ivacaftor glycoside in the composition will also depend upon the particular ivacaftor glycoside in the composition.

Other pharmaceutically acceptable carriers, adjuvants and vehicles that can be used in the compositions of this invention include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives can also be advantageously used to enhance delivery of agents described herein.

In some embodiments, compositions comprising an ivacaftor glycoside described herein, or a pharmaceutically acceptable salt thereof, can also include one or more other therapeutic agents, e.g., in combination. When the compositions of this invention comprise a combination, the agents should be present at dosage levels of between about 1 to 100%, and more preferably between about 5% to about 95% of the dosage normally administered in a monotherapy regimen.

The compositions described herein can, for example, be administered by injection, intravenously, intraarterially, intraocularly, intravitreally, subdermally, orally, buccally, nasally, transmucosally, topically, in an ophthalmic preparation, or by inhalation, with a dosage ranging from about 0.5 mg/kg to about 100 mg/kg of body weight or, alternatively, in a dosage ranging from about 1 mg/dose to about 1000 mg/dose, every 4 to 120 hours, or according to the requirements of the particular drug. Typically, the compositions will be administered from about 1 to about 6 (e.g., 1, 2, 3, 4, 5 or 6) times per day or, alternatively, as an infusion (e.g., a continuous infusion). The amount of active ingredient that can be combined with a carrier material to produce a single dosage form will vary depending upon the host treated and the particular mode of administration. A typical preparation will contain from about 1% to about 95%, from about 2.5% to about 95% or from about 5% to about 95% of an ivacaftor glycoside (w/w). Alternatively, a preparation can contain from about 20% to about 80% of an ivacaftor glycoside (w/w).

Doses lower or higher than those recited above may be required. Specific dosage and treatment regimens for any particular patient will depend upon a variety of factors, including the activity of the specific agent employed, the age, body weight, general health status, sex, diet, time of administration, rate of excretion, drug combination, the severity and course of the disease, condition or symptoms, the patient's disposition to the disease, condition or symptoms, and the judgment of the treating physician.

“Treating,” as used herein, refers to taking steps to deliver a therapy to a subject, such as a mammal, in need thereof (e.g., as by administering to a mammal one or more therapeutic agents). “Treating” includes inhibiting the disease or condition (e.g., as by slowing or stopping its progression or causing regression of the disease or condition), and relieving the symptoms resulting from the disease or condition.

“A therapeutically effective amount” is an amount effective, at dosages and for periods of time necessary, to achieve a desired therapeutic result (e.g., treatment, healing, inhibition or amelioration of physiological response or condition, etc.). The full therapeutic effect does not necessarily occur by administration of one dose, and may occur only after administration of a series of doses. Thus, a therapeutically effective amount may be administered in one or more administrations. A therapeutically effective amount may vary according to factors such as disease state, age, sex, and weight of a mammal, mode of administration and the ability of a therapeutic, or combination of therapeutics, to elicit a desired response in an individual.

An effective amount of an agent to be administered can be determined by a clinician of ordinary skill using the guidance provided herein and other methods known in the art. For example, suitable dosages can be from about 0.001 mg/kg to about 100 mg/kg, from about 0.01 mg/kg to about 100 mg/kg, from about 0.01 mg/kg to about 10 mg/kg, from about 0.01 mg/kg to about 1 mg/kg body weight per treatment. Determining the dosage for a particular agent, subject and disease is well within the abilities of one of skill in the art. Preferably, the dosage does not cause adverse side effects or produces minimal adverse side effects.

As used herein, “subject” includes humans, domestic animals, such as laboratory animals (e.g., dogs, monkeys, pigs, rats, mice, etc.), household pets (e.g., cats, dogs, rabbits, etc.) and livestock (e.g., pigs, cattle, sheep, goats, horses, etc.), and non-domestic animals. In some embodiments, a subject is a human. “Subject” and “patient” are used interchangeably herein.

An ivacaftor glycoside described herein, or a pharmaceutically acceptable salt thereof, can be administered via a variety of routes of administration, including, for example, oral, dietary, topical, transdermal, rectal, parenteral (e.g., intra-arterial, intravenous, intramuscular, subcutaneous injection, intradermal injection), intravenous infusion and inhalation (e.g., intrabronchial, intranasal or oral inhalation, intranasal drops) routes of administration, depending on the ivacaftor glycoside and the particular disease to be treated. Administration can be local or systemic as indicated. The preferred mode of administration can vary depending on the particular ivacaftor glycoside chosen.

Certain methods further specify a delivery route such as intravenous, intramuscular, subcutaneous, rectal, intranasal, pulmonary, or oral.

An ivacaftor glycoside described herein, or a pharmaceutically acceptable salt thereof, can also be administered in combination with one or more other therapies (e.g., radiation therapy, a chemotherapy, such as a chemotherapeutic agent; an immunotherapy, such as an immunotherapeutic agent). When administered in a combination therapy, the ivacaftor glycoside, or pharmaceutically acceptable salt thereof, can be administered before, after or concurrently with the other therapy (e.g., radiation therapy, an additional agent(s)). When co-administered simultaneously (e.g., concurrently), the ivacaftor glycoside, or pharmaceutically acceptable salt thereof, and other therapy can be in separate formulations or the same formulation. Alternatively, the ivacaftor glycoside, or pharmaceutically acceptable salt thereof, and other therapy can be administered sequentially, as separate compositions, within an appropriate time frame as determined by a skilled clinician (e.g., a time sufficient to allow an overlap of the pharmaceutical effects of the therapies).

In some embodiments, a method described herein further includes administering to the subject a therapeutically effective amount of an additional therapy (e.g., an additional therapeutic agent, such as KALYDECO®, ORKAMBI®, SYMDECO®, or TRIKAFTA®).

### SUMMARY

Ivacaftor is a lipophilic, low solubility, high impact therapeutic that could benefit from modification by glycosylation.

There is a need for CFTR modulators with improved aqueous solubility and with different PK/PD profiles to provide potential improvements in potency towards modulating the activity of the CFTR protein and enhanced therapeutic effects on CF and CFTR-related diseases.

### EXEMPLIFICATION

### Example 1: Establishment of a Glycosyltransferase (GT) Library and Cell Lysate-Based Assay to Identify Drug-Modifying Glycosyltransferases

Although GTs are one of the largest enzyme families in nature, the natural substrate(s) of the majority of GTs is unknown. Therefore, to identify GTs that can use a non-native substrate such as ivacaftor is a nontrivial effort. A screening strategy was designed to address this need. The phylogenetic method was utilized to select a set of enzymes representing the structural and functional biodiversity of a desired functional GT class, uridine diphosphate (UDP) glycosyltransferases (UGTs), across different kingdoms and species. Based on the bioinformatics analysis, 328 UGTs were selected, including enzymes from different species of bacteria, fungus, plants, and human. To establish the GT library, the cDNA of the selected UGTs were produced by either nucleotide synthesis or by RT-PCR from the RNA of tissues expressing the UGTs. Each of the resulting UGT gene cDNA was cloned into the yeast TEF-promoter expression plasmid p426-TEF. The plasmids were individually transformed into wild-type yeast (Saccharomyces cerevisiae) strain BY4743. After auxotrophic selection, the yeast colonies expressing the recombinant UGT proteins were cultured, harvested, and lysed by CelLytic Y cell lysis reagent (Sigma-Aldrich). A cell-free cell lysate-based glycosylation assay was designed to screen for UGTs that are able to glycosylate the target substrate (see below for details). All UGTs were assayed in parallel on 96-well plates to allow for high throughput screening. The drug-modifying UGTs can be identified by the appearance of new peaks in HPLC analysis. The characteristics of the novel drug glycosides can be evaluated further by specialized assays.

### Example 2: Synthesis of Ivacaftor-17-O-D-Glucoside and Ivacaftor-17-di-O-D-Glucoside Using the Cell Lysate-Based Assay

A GT library made according to Example 1 was screened to identify enzymes able to catalyze regiospecific glycosylation of ivacaftor when UDP-glucose was used as the sugar donor. Ivacaftor (final concentration=50 μM) was added to each well of a 96-well microtiter plate containing a unique UGT enzyme in reaction mixture (50 mM Tris, pH 8.0, 10 mM UDP-glucose, and 20 μL recombinant UGT-expressing yeast cell lysate) and the reaction (total volume 100 μL) was allowed to proceed for 24 hours at 30 C, followed by termination of the modification reaction by quenching with 100 μL methanol. As a negative control, a reaction with the lysate of yeast harboring p426-TEF empty vector was carried out. The presence of the desired glycosylated product was determined by subjecting the contents of each well to HPLC analysis.

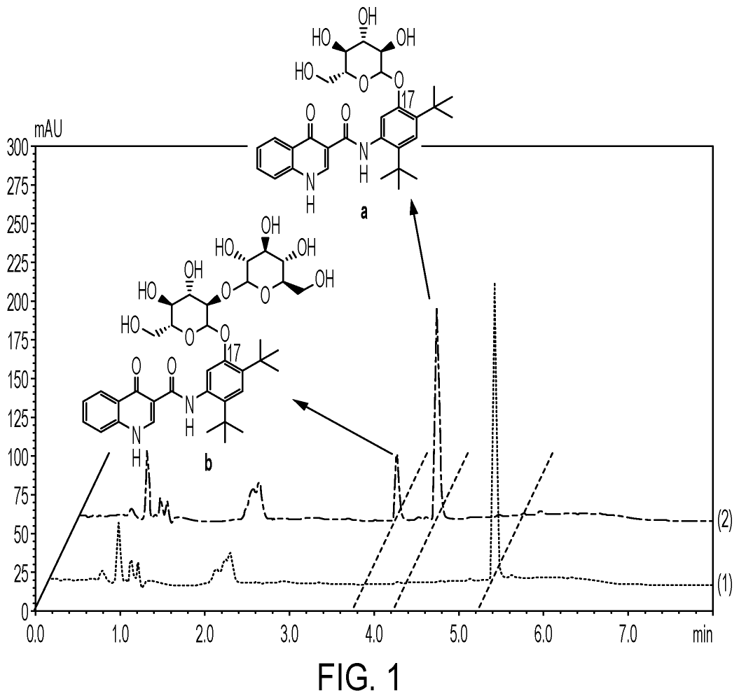

From the screen, four UGTs were able to modify ivacaftor when UDP-glucose was used as the sugar donor. The overall conversion rates are: 99% for SEQ ID NO: 2, 97% for SEQ ID NO: 4, 96% for SEQ ID NO: 1, 40% for SEQ ID NO: 3. Among the four UGTs, SEQ ID NO: 2, SEQ ID NO: 4 and SEQ ID NO: 1 can produce both the monosaccharide ivacaftor-17-O-D-glucoside (FIG. 1, chromatogram peak a) and the disaccharide ivacaftor-17-di-O-D-glucoside (FIG. 1, chromatogram peak b). SEQ ID NO: 3 can produce ivacaftor-17-O-D-glucoside only.

The chemical identity of the ivacaftor glycosides was confirmed by LC-MS analysis: For a: m/z=553.30 [M−H]⁻; For b: m/z=715.40 [M−H]⁻.

The chemical identity of the ivacaftor glycosides produced using SEQ ID NO: 2 was further confirmed by nuclear magnetic resonance (NMR) analyses: For a: ¹H NMR (DMSO-d₆, 500 MHz), δ 12.34 (1H, s), 8.84 (1H, s), 8.28 (1H, d, J=8.0 Hz), 7.68 (1H, s), 7.67 (1H, s), 7.48 (1H, s), 7.39 (1H, m), 7.22 (1H, s), 4.85 (1H, d, J=7.5 Hz), 3.68 (1H, m), 3.48 (1H, m), 3.24 (2H, m), 3.17 (1H, m), 1.40 (9H, s), 1.38 (9H, s); ¹³C NMR (DMSO-d₆, 125 MHz), δ 175.8, 164.1, 153.4, 146.5, 142.1, 134.3, 134.3, 133.1, 131.9, 126.5, 125.4, 124.4, 123.6, 121.3, 114.3, 110.2, 99.9, 77.4, 77.3, 73.7, 69.8, 60.9, 34.6, 34.1, 30.4, 30.0; For b: ¹H NMR (DMSO-d₆, 500 MHz), δ 12.64 (1H, s), 8.83 (1H, s), 8.25 (1H, dd, J=8.0, 0.5 Hz), 7.64 (1H, d, J=8.0 Hz), 7.59 (1H, t, J=8.0 Hz), 7.40 (1H, s), 7.31 (1H, t, J=7.5 Hz), 7.21 (1H, s), 5.05 (1H, d, J=8.0 Hz), 4.72 (1H, d, J=8.0 Hz), 3.85 (1H, t, J=8.5 Hz), 3.67 (1H, d, J=11.5 Hz), 3.56 (2H, d, J=9.0 Hz), 3.22 (2H, dd, J=18.5, 9.5 Hz), 3.12 (1H, t, J=8.5 Hz), 3.03 (2H, m), 2.97 (1H, t, J=8.0 Hz), 1.39 (9H, s), 1.37 (9H, s); ¹³C NMR (DMSO-d₆, 100 MHz), δ 175.6, 165.6, 153.2, 149.2, 145.7, 134.9, 134.9, 133.5, 131.3, 127.4, 125.6, 124.0, 124.0, 123.9, 114.7, 109.9, 103.1, 98.1, 78.7, 77.9, 77.6, 77.4, 76.8, 74.9, 70.6, 70.2, 61.6, 61.3, 35.0, 34.6, 30.9, 30.6.

The sequence of the enzymes identified as SEQ ID NOs.: 1-4 are disclosed herein in the sequences section.

### Example 3: Synthesis of ivacaftor-17-O-D-glucoside, ivacaftor-17-di-O-D-glucoside, and ivacaftor-17-tri-O-D-glucoside Using Purified Recombinant Glycosyltransferases

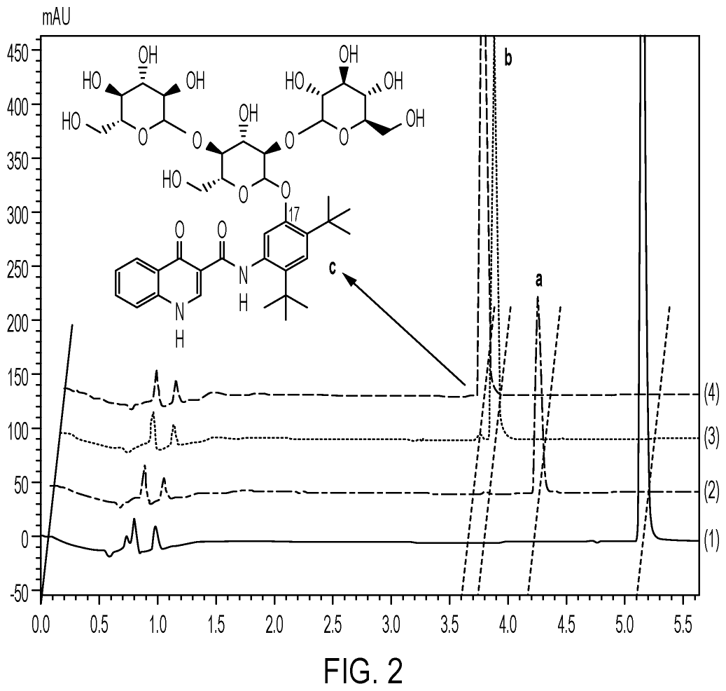

While a yeast cell lysate-based glycosylation assay is instrumental in initial screening efforts, one approach to producing larger amounts of ivacaftor glucosides is to use finely controlled enzyme concentrations during synthesis. To that end, two UGT genes identified in Example 2 (SEQ ID NO: 1 and 2) containing a metal-affinity purification tag at the C-terminus were transformed into BL21(DE3) Escherichia coli cells. Cells were grown at 37° C. until the cultures reached an optical density (OD600) of 0.5-0.8. Then, protein over-expression was induced with 1 mM isopropyl β-D-1-thiogalactopyranoside (IPTG) at 18° C. The culture was grown overnight for 16 hours and then harvested. Desired proteins were purified from the harvested cells using either free nickel-IDA resin or magnetic nickel-charged agarose beads. Ivacaftor glucoside synthesis using the purified recombinant enzymes was performed at volumes ranging from 10-75 mL. Ivacaftor (final concentration 0.1-0.2 mg/ml) was added to the reaction mixture (final concentrations of 50 mM HEPES, 50 mM KCl, pH 7.5, 2 mM UDP-glucose, 1 uM UGT), and the reaction was allowed to proceed for 1-3 days at 37° C. The reaction was terminated by adding 1 reaction volume of ice-cold methanol. The reaction was then incubated at 90° C. to ensure that the enzyme was adequately denatured. The presence of the desired glycosylated product(s) was determined by HPLC analysis (FIG. 2). From these reactions, SEQ ID NO: 1 and 2 can produce both the monosaccharide ivacaftor-17-O-D-glucoside (FIG. 2, chromatogram peak a) and the disaccharide ivacaftor-17-di-O-D-glucoside (FIG. 2, chromatogram peak b). SEQ ID NO: 2 can also produce the trisaccharide ivacaftor-17-tri-O-D-glucoside (FIG. 2, chromatogram peak c).

The chemical identity of the ivacaftor glycosides was confirmed by LC-MS analysis: For a: m/z=555.43 [M+H]*; For b: m/z=717.17 [M+H]*; For c: m/z=879.21 [M+H]+.

The chemical identity of the trisaccharide ivacaftor-17-tri-O-D-glucoside produced using SEQ ID NO: 2 (FIG. 2, chromatogram peak c) was further confirmed by nuclear magnetic resonance (NMR) analyses: ¹H NMR (DMSO-d₆, 500 MHz), δ 12.29 (1H, s), 8.84 (1H, s), 8.29 (1H, d, J=8.0 Hz), 7.69 (2H, d, J=3.55 Hz), 7.42 (1H, s), 7.40 (1H, dd, J=8.0, 4.0 Hz), 7.23 (1H, s), 5.25 (1H, d, J=4.1 Hz), 5.08 (1H, d, J=3.6 Hz), 5.05 (1H, d, J=7.7 Hz), 4.99 (2H, m), 4.94 (1H, s), 4.88 (2H, m), 4.80 (1H, d, J=7.7 Hz), 4.63 (1H, m), 4.57 (1H, t, J=6.0 Hz), 4.28 (1H, d, J=7.9 Hz), 4.26 (1H, t, J=5.5 Hz), 4.03 (1H, t, J=8.1 Hz), 3.77 (2H, m), 3.67 (4H, m), 3.15 (4H, m), 3.06 (3H, m), 2.97 (4H, m), 1.40 (9H, s), 1.37 (9H, s); ¹³C NMR (DMSO-d₆, 125 MHz), δ 175.7, 164.2, 152.9, 146.7, 142.4, 134.4, 134.2, 133.2, 131.8, 126.5, 125.3, 124.2, 123.6, 121.6, 114.1, 110.1, 103.2, 101.7, 97.7, 80.0, 77.0, 76.8, 76.4, 76.3, 76.1, 76.0, 75.3, 74.2, 73.3, 70.6, 69.9, 61.6, 60.9, 60.0, 34.6, 34.1, 30.4, 30.2.

### Example 4: Synthesis of ivacaftor-17-O-D-galactoside Using the Cell Lysate-Based Assay

A GT library made according to Example 1 was screened to identify enzymes able to catalyze regiospecific glycosylation of ivacaftor when UDP-galactose was used as the sugar donor. Ivacaftor (final concentration=50 μM) was added to each well of a 96-well microtiter plate containing a unique UGT enzyme in reaction mixture (50 mM Tris, pH 8.0, 2 mM UDP-galactose and 20 μL recombinant UGT-expressing yeast cell lysate) and the reaction (total volume 100 μL) was allowed to proceed for 5 hours at 30′C, followed by termination of the modification reaction by quenching with 100 μL methanol. As a negative control, a reaction with the lysate of yeast harboring p426-TEF empty vector was carried out. The presence of the desired glycosylated product was determined by subjecting the contents of each well to HPLC analysis.

From the screen, four UGTs were able to modify ivacaftor when UDP-galactose was used as the sugar donor. The overall conversion rates are: 23% for SEQ ID NO: 2, 28% for SEQ ID NO: 4, 6% for SEQ ID NO: 1, 5% for SEQ ID NO: 3. All four UGTs can produce monosaccharide ivacaftor-17-O-D-galactoside.

The chemical identity of the ivacaftor glycosides was confirmed by LC-MS analysis: m/z=555.29 [M+H]⁺.

### Example 5: Synthesis of ivacaftor-17-O-D-xyloside and ivacaftor-17-di-O-D-xyloside Using the Cell Lysate-Based Assay

A GT library made according to Example 1 was screened to identify enzymes able to catalyze regiospecific glycosylation of ivacaftor when UDP-xylose was used as the sugar donor. Ivacaftor (final concentration=50 μM) was added to each well of a 96-well microtiter plate containing a unique UGT enzyme in reaction mixture (50 mM Tris, pH 8.0, 2 mM UDP-xylose and 20 μL recombinant UGT-expressing yeast cell lysate) and the reaction (total volume 100 μL) was allowed to proceed for 5 hours at 30° C., followed by termination of the modification reaction by quenching with 100 μL methanol. As a negative control, a reaction with the lysate of yeast harboring p426-TEF empty vector was carried out. The presence of the desired glycosylated product was determined by subjecting the contents of each well to HPLC analysis.

From the screen, four UGTs were able to modify ivacaftor when UDP-xylose was used as the sugar donor. The overall conversion rates are: 10% for SEQ ID NO: 2, 66% for SEQ ID NO: 4, 46% for SEQ ID NO: 1, 5% for SEQ ID NO: 3. Among the four UGTs, SEQ ID NO: 2, SEQ ID NO: 4 and SEQ ID NO: 1 can produce both the monosaccharide ivacaftor-17-O-D-xyloside (d) and the disaccharide ivacaftor-17-di-O-D-xyloside (e). SEQ ID NO: 3 can produce ivacaftor-17-O-D-xyloside only.

The chemical identity of the ivacaftor glycosides was confirmed by LC-MS analysis: For d: m/z=525.28 [M+H]*; For e: m/z=657.67 [M+H]⁺.

### Example 6: Synthesis of ivacaftor-17-O-N-acetylglucosamide Using the Cell Lysate-Based Assay

A GT library made according to Example 1 was screened to identify enzymes able to catalyze regiospecific glycosylation of ivacaftor when UDP-N-acetylglucosamine was used as the sugar donor. Ivacaftor (final concentration=50 μM) was added to each well of a 96-well microtiter plate containing a unique UGT enzyme in reaction mixture (50 mM Tris, pH 8.0, 2 mM UDP-N-acetylglucosamine and 20 μL recombinant UGT-expressing yeast cell lysate) and the reaction (total volume 100 μL) was allowed to proceed for 5 hours at 30 C, followed by termination of the modification reaction by quenching with 100 μL methanol. As a negative control, a reaction with the lysate of yeast harboring p426-TEF empty vector was carried out. The presence of the desired glycosylated product was determined by subjecting the contents of each well to HPLC analysis.

From the screen, only one UGT was able to modify ivacaftor when UDP-N-acetylglucosamine was used as the sugar donor. The overall conversion rate is: 1.4% for SEQ ID NO: 2. SEQ ID NO: 2 can produce the monosaccharide ivacaftor-17-O-N-acetylglucosamide.

The chemical identity of the ivacaftor glycoside was confirmed by LC-MS analysis: m/z=596.36 [M+H]⁺.

### Example 7: Comparison of the Water Solubility of ivacaftor and ivacaftor-17-O-D-glucoside

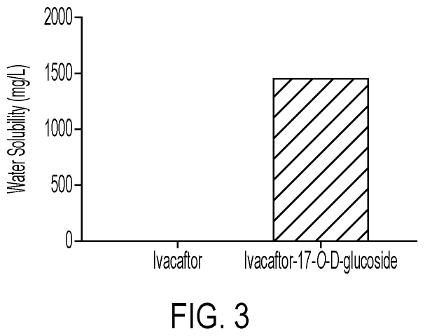

The water solubility of ivacaftor and ivacaftor-17-O-D-glucoside was investigated by suspending excess amounts of the two compounds in 200 μl of distilled water in a microcentrifuge tube at 25° C. for 12 h. Afterwards, each sample was centrifuged at 12,000×g for 20 min. The supernatant of each sample was then filtered through a 0.45-μm membrane filter and the concentration of the compound in the supernatant, which is defined as the water-soluble component, was measured by its absorbance at 300 nm using HPLC, and its absolute solubility was calculated in reference to the concentration-absorbance standard curve. As shown in FIG. 3, the water solubility of ivacaftor was determined to be 0.1 mg/L, whereas that of ivacaftor-17-O-D-glucoside was 1,455 mg/L, which is 14,550 times higher.

### REFERENCES

1. Abel, Mark, Roman Szweda, Daniel Trepanier, Randall W. Yatscoff, and Robert T. Foster. 2007. Rapamycin carbohydrate derivatives. USPTO 7,160,867 B2. US Patent, filed May 13, 2004, and issued Jan. 9, 2007.
2. “About Cystic Fibrosis.” n.d. Accessed Jan. 13, 2020a. https://www.cff.org/What-is-CF/About-Cystic-Fibrosis/.
3. “What is Cystic Fibrosis?” About Cystic Fibrosis. n.d. Accessed Jan. 13, 2020b. https://www.cff.org/What-is-CF/About-Cystic-Fibrosis/.
4. Arts, Ilja C. W., Aloys L. A. Sesink, Maria Faassen-Peters, and Peter C. H. Hollman. 2004. “The Type of Sugar Moiety Is a Major Determinant of the Small Intestinal Uptake and Subsequent Biliary Excretion of Dietary Quercetin Glycosides.” The British Journal of Nutrition 91 (6): 841-47.
5. Berman, H. M. 2000. “The Protein Data Bank.” Nucleic Acids Research. https://doi.org/10.1093/nar/28.1.235.
6. Bombieri, C., M. Claustres, K. De Boeck, N. Derichs, J. Dodge, E. Girodon, I. Sermet, et al. 2011. “Recommendations for the Classification of Diseases as CFTR-Related Disorders.” Journal of Cystic Fibrosis: Official Journal of the European Cystic Fibrosis Society 10 Suppl 2 (June): S86-102.
7. Bonina, Francesco, Carmelo Puglia, Maria Grazia Rimoli, Daniela Melisi, Giampiero Boatto, Maria Nieddu, Antonio Calignano, Giovanna La Rana, and Paolo De Caprariis. 2003. “Glycosyl Derivatives of Dopamine and L-Dopa as Anti-Parkinson Prodrugs: Synthesis, Pharmacological Activity and in Vitro Stability Studies.” Journal of Drug Targeting 11 (1): 25-36.
8. Breton, Christelle, Sylvie Fournel-Gigleux, and Monica M. Palcic. 2012. “Recent Structures, Evolution and Mechanisms of Glycosyltransferases.” Current Opinion in Structural Biology 22 (5): 540-49.
9. Cil, Onur, Puay-Wah Phuan, Sujin Lee, Joseph Tan, Peter M. Haggie, Marc H. Levin, Liang Sun, Jay R. Thiagarajah, Tonghui Ma, and Alan S. Verkman. 2016. “CFTR Activator Increases Intestinal Fluid Secretion and Normalizes Stool Output in a Mouse Model of Constipation.” Cellular and Molecular Gastroenterology and Hepatology 2 (3): 317-27.
10. Cystic Fibrosis Foundation. 2018. “Cystic Fibrosis Foundation Patient Registry: 2018 Annual Data Report.” https://www.cff.org/Research/Researcher-Resources/Patient-Registry/2018-Patient-Registry-Annual-Data-Report.pdf.
11. De Bruyn, Frederik, Jo Maertens, Joeri Beauprez, Wim Soetaert, and Marjan De Mey. 2015. “Biotechnological Advances in UDP-Sugar Based Glycosylation of Small Molecules.” Biotechnology Advances 33 (2): 288-302.
12. Desai, Sameer, Hubert Wong, Jenna Sykes, Anne L. Stephenson, Joel Singer, and Bradley S. Quon. 2018. “Clinical Characteristics and Predictors of Reduced Survival for Adult-Diagnosed Cystic Fibrosis. Analysis of the Canadian CF Registry.” Annals of the American Thoracic Society 15 (10): 1177-85.
13. Eckford, Paul D. W., Canhui Li, Mohabir Ramjeesingh, and Christine E. Bear. 2012. “Cystic Fibrosis Transmembrane Conductance Regulator (CFTR) Potentiator VX-770 (Ivacaftor) Opens the Defective Channel Gate of Mutant CFTR in a Phosphorylation-Dependent but ATP-Independent Manner.” Journal of Biological Chemistry. https://doi.org/10.1074/jbc.m 112.393637.
14. Fernández, C., O. Nieto, E. Rivas, G. Montenegro, J. A. Fontenla, and A. Fernández-Mayoralas. 2000. “Synthesis and Biological Studies of Glycosyl Dopamine Derivatives as Potential Antiparkinsonian Agents.” Carbohydrate Research 327 (4): 353-65.
15. Flores, Alyssa M., Scott D. Casey, Christian M. Felix, Puay W. Phuan, A. S. Verkman, and Marc H. Levin. 2016. “Small-Molecule CFTR Activators Increase Tear Secretion and Prevent Experimental Dry Eye Disease.” FASEB Journal: Official Publication of the Federation of American Societies for Experimental Biology 30 (5): 1789-97.
16. Frossard, Jean-Louis, Jean-Marc Dumonceau, Laurent Spahr, Emile Giostra, and Antoine Hadengue. 2007. “Coincidental CFTR Mutation and Sjögren Syndrome Revealing a Pancreatic Disease.” Pancreas 35 (1): 94-95.
17. Gantt, Richard W., Pauline Peltier-Pain, and Jon S. Thorson. 2011. “Enzymatic Methods for Glyco(diversification/randomization) of Drugs and Small Molecules.” Natural Product Reports 28 (11): 1811-53.
18. Gu, Xiangying, Lin Chen, Xin Wang, Xiao Liu, Qidong You, Wenwei Xi, Li Gao, et al. 2014. “Direct Glycosylation of Bioactive Small Molecules with Glycosyl Iodide and Strained Olefin as Acid Scavenger.” The Journal of Organic Chemistry 79 (3): 1100-1110.
19. Habib, Al-Rahim R., Majid Kajbafzadeh, Sameer Desai, Connie L. Yang, Kate Skolnik, and Bradley S. Quon. 2019. “A Systematic Review of the Clinical Efficacy and Safety of CFTR Modulators in Cystic Fibrosis.” Scientific Reports 9 (1): 7234.
20. Hadida-Ruah, Sara, Anna Hazelwood, Peter Grootenhuis, Fred Van Goor, Ashvani Singh, Jinglan Zhou, and Jason McCartney. 2009. Modulators of ATP-binding cassette transporters. USPTO 7,495,103 B2. US Patent, filed Jun. 24, 2005, and issued Feb. 24, 2009.
21. Hardman, Janee' M., Robert T. Brooke, and Brandon J. Zipp. 2017. “Cannabinoid Glycosides: In Vitro Production of a New Class of Cannabinoids with Improved Physicochemical Properties.” bioRxiv. https://doi.org/10.1101/104349.
22. Jordan, Cameron L., Terry L. Noah, and Marianna M. Henry. 2016. “Therapeutic Challenges Posed by Critical Drug-Drug Interactions in Cystic Fibrosis.” Pediatric Pulmonology 51 (S44): S61-70.
23. Krebber S., Claus, Christopher Davis, Stephen Delcardayre, Sergey A. Selifonov, and Russell Howard. 2001. Evolution and use of enzymes for combinatorial and medicinal chemistry. WIPO WO/2001/012817 A1. World Patent, filed Aug. 11, 2000, and published Feb. 22, 2001.
24. Křen, Vladimir. 2008. “Glycoside vs. Aglycon: The Role of Glycosidic Residue in Biological Activity.” Glycoscience, 2589-2644.
25. Kren, V., and L. Martínková. 2001. “Glycosides in Medicine: ‘The Role of Glycosidic Residue in Biological Activity.’”Current Medicinal Chemistry 8 (11): 1303-28.
26. Lairson, L. L., B. Henrissat, G. J. Davies, and S. G. Withers. 2008. “Glycosyltransferases: Structures, Functions, and Mechanisms.” Annual Review of Biochemistry 77 (1): 521-55.
27. Levin, Marc H., and A. S. Verkman. 2005. “CFTR-Regulated Chloride Transport at the Ocular Surface in Living Mice Measured by Potential Differences.” Investigative Opthalmology & Visual Science 46 (4): 1428.
28. Lin, Yih-Shyan, Rudeewan Tungpradit, Supachok Sinchaikul, Feng-Ming An, Der-Zen Liu, Suree Phutrakul, and Shui-Tein Chen. 2008. “Targeting the Delivery of Glycan-Based Paclitaxel Prodrugs to Cancer Cells via Glucose Transporters.” Journal of Medicinal Chemistry 51 (23): 7428-41.
29. Liu, Der-Zen, Supachok Sinchaikul, Peddiahgari Vasu Govardhana Reddy, Meng-Yang Chang, and Shui-Tein Chen. 2007. “Synthesis of 2′-Paclitaxel Methyl 2-Glucopyranosyl Succinate for Specific Targeted Delivery to Cancer Cells.” Bioorganic & Medicinal Chemistry Letters. https://doi.org/10.1016/j.bmdl.2006.11.008.
30. Lombard, Vincent, Hemalatha Golaconda Ramulu, Elodie Drula, Pedro M. Coutinho, and Bernard Henrissat. 2014. “The Carbohydrate-Active Enzymes Database (CAZy) in 2013.” Nucleic Acids Research 42 (D1): D490-95.
31. Mikuni, Katsuhiko, Katsuyoshi Nakanishi, Koji Hara, Kozo Hara, Wakao Iwatani, Tetsuya Amano, Kosho Nakamura, Yoshinori Tsuchiya, Hiroshi Okumoto, and Tadakatsu Mandai. 2008. “In Vivo Antitumor Activity of Novel Water-Soluble Taxoids.” Biological & Pharmaceutical Bulletin 31 (6): 1155-58.
32. Morgan, Adam J. 2012. Deuterated derivatives of ivacaftor. WO/2012/158885 A1.
33. Munoz, Benito, Daniel Parks, and Cecilia M. Bastos. 2017. Silicon atoms containing ivacaftor analogues. WO/2017/177124 A1.
34. Noone, P. G., and M. R. Knowles. 2001. “‘CFTR-Opathies’: Disease Phenotypes Associated with Cystic Fibrosis Transmembrane Regulator Gene Mutations.” Respiratory Research 2 (6): 328-32.
35. Olthof, Margreet R., Peter C. H. Hollman, Tom B. Vree, and Martijn B. Katan. 2000. “Bioavailabilities of Quercetin-3-Glucoside and Quercetin-4′-Glucoside Do Not Differ in Humans.” The Journal of Nutrition 130 (5): 1200-1203.
36. Peltier-Pain, Pauline, Shannon C. Timmons, Agnes Grandemange, Etienne Benoit, and Jon S. Thorson. 2011. “Warfarin Glycosylation Invokes a Switch from Anticoagulant to Anticancer Activity.” ChemMedChem 6 (8): 1347-50.
37. Polt, Robin, Muthu Dhanasekaran, and Charles M. Keyari. 2005. “Glycosylated Neuropeptides: A New Vista for Neuropsychopharmacology?” Medicinal Research Reviews 25 (5): 557-85.
38. Rafeeq, Misbahuddin M., and Hussam Aly Sayed Murad. 2017. “Cystic Fibrosis: Current Therapeutic Targets and Future Approaches.” Journal of Translational Medicine 15 (1): 84.
39. Rowe, Steven M., Stacey Miller, and Eric J. Sorscher. 2005. “Cystic Fibrosis.” The New England Journal of Medicine 352 (19): 1992-2001.
40. Schmid, Jochen, Dominik Heider, Norma J. Wendel, Nadine Sperl, and Volker Sieber. 2016. “Bacterial Glycosyltransferases: Challenges and Opportunities of a Highly Diverse Enzyme Class Toward Tailoring Natural Products.” Frontiers in Microbiology 7 (February): 182.
41. Sloane, Peter A., Suresh Shastry, Andrew Wilhelm, Clifford Courville, Li Ping Tang, Kyle Backer, Elina Levin, et al. 2012. “A Pharmacologic Approach to Acquired Cystic Fibrosis Transmembrane Conductance Regulator Dysfunction in Smoking Related Lung Disease.” PloS One 7 (6): e39809.
42. Solomon, George M., S. Vamsee Raju, Mark T. Dransfield, and Steven M. Rowe. 2016. “Therapeutic Approaches to Acquired Cystic Fibrosis Transmembrane Conductance Regulator Dysfunction in Chronic Bronchitis.” Annals of the American Thoracic Society 13 Suppl 2 (April): S169-76.
43. Takechi, M., and Y. Tanaka. 1994. “Structure-Activity Relationships of Synthetic Digitoxigenyl Glycosides.” Phytochemistry 37 (5): 1421-23.
44. Terao, Junji, Sachiyo Yamaguchi, Mutsuko Shirai, Mariko Miyoshi, Jae-Hak Moon, Syunji Oshima, Takahiro Inakuma, Tojiro Tsushida, and Yoji Kato. 2001. “Protection by Quercetin and Quercetin 3-O-β-D-Glucuronide of Peroxynitrite-Induced Antioxidant Consumption in Human Plasma Low-Density Lipoprotein.” Free Radical Research. https://doi.org/10.1080/10715760100301421.
45. Torrens-Spence, Michael P., Tomáš Pluskal, Fu-Shuang Li, Valentina Carballo, and Jing-Ke Weng. 2018. “Complete Pathway Elucidation and Heterologous Reconstitution of Rhodiola Salidroside Biosynthesis.” Molecular Plant 11 (1): 205-17.
46. Van Goor, Fredrick, Sabine Hadida, Peter D. J. Grootenhuis, Bill Burton, Dong Cao, Tim Neuberger, Amanda Turnbull, et al. 2009. “Rescue of CF Airway Epithelial Cell Function in Vitro by a CFTR Potentiator, VX-770.” Proceedings of the National Academy of Sciences of the United States of America 106 (44): 18825-30.
47. “Vertex Pharmaceuticals. Kalydeco (ivacaftor) [package Insert].” 2019. U.S. Food and Drug Administration Website. Apr. 29, 2019. https://www.accessdata.fda.gov/drugsatfda_docs/label/2019/203188s029,207925s008lbl.pdf.
48. “Vertex Pharmaceuticals. Orkambi (ivacaftor; Lumacaftor) [package Insert].”2018. U.S. Food and Drug Administration Website. Aug. 15, 2018. https://www.accessdata.fda.gov/drugsatfda_docs/label/2018/206038s010lbl.pdf.
49. “Vertex Pharmaceuticals. Symdeko (ivacaftor; Tezacaftor) [package Insert].”2019. U.S. Food and Drug Administration Website. Dec. 27, 2019. https://www.accessdata.fda.gov/drugsatfda_docs/label/2019/210491s005lbl.pdf.
50. “Vertex Pharmaceuticals. Trikafta (ivacaftor; Elexacaftor) [package Insert].”2019. U.S. Food and Drug Administration Website. Oct. 21, 2019. https://www.accessdata.fda.gov/drugsatfda_docs/label/2019/212273s000lbl.pdf.
51. Xiao, Jianbo, Hui Cao, Yuanfeng Wang, Jinyao Zhao, and Xinlin Wei. 2009. “Glycosylation of Dietary Flavonoids Decreases the Affinities for Plasma Protein.” Journal of Agricultural and Food Chemistry 57 (15): 6642-48.
52. Xie, Kebo, Ridao Chen, Dawei Chen, Jianhua Li, Ruishan Wang, Lin Yang, and Jungui Dai. 2017. “Enzymatic N-Glycosylation of Diverse Arylamine Aglycones by a Promiscuous Glycosyltransferase from Carthamus Tinctorius.” Advanced Synthesis & Catalysis 359 (4): 603-8.
53. Yonekura-Sakakibara, Keiko, and Kousuke Hanada. 2011. “An Evolutionary View of Functional Diversity in Family 1 Glycosyltransferases.” The Plant Journal: For Cell and Molecular Biology 66 (1): 182-93.
54. Zhang, Yujiao, Kebo Xie, Aijing Liu, Ridao Chen, Dawei Chen, Lin Yang, and Jungui Dai. 2019. “Enzymatic Biosynthesis of Benzylisoquinoline Alkaloid Glycosides via Promiscuous Glycosyltransferases from Carthamus Tinctorius.” Chinese Chemical Letters 30 (2): 443-46.
55. Zhu, Xiangming, and Richard R. Schmidt. 2009. “New Principles for Glycoside-Bond Formation.” Angewandte Chemie 48 (11): 1900-1934.
56. Zipp, Brandon Joel, Janee M. Hardman, and Robert T. Brooke. 2018. Cannabinoid glycoside prodrugs and methods of synthesis. USPTO US 20180264122 A1. US Patent, published Sep. 20, 2018.
57. Bairoch, A., PROSITE: a dictionary of sites and patterns in proteins. Nucleic acids research, 1991. 19 Suppl(Suppl): p. 2241-2245.
58. Hughes, J. and M. A. Hughes, Multiple secondary plant product UDP-glucose glucosyltransferase genes expressed in cassava (Manihot esculenta Crantz) cotyledons. DNA Seq, 1994. 5(1): p. 41-9.
59. Yamazaki, M., et al., Molecular cloning and biochemical characterization of a novel anthocyanin 5-O-glucosyltransferase by mRNA differential display for plant forms regarding anthocyanin. J Biol Chem, 1999. 274(11): p. 7405-11.
60. Hans, J., W. Brandt, and T. Vogt, Site-directed mutagenesis and protein 3D-homology modelling suggest a catalytic mechanism for UDP-glucose-dependent betanidin 5-O-glucosyltransferase from Dorotheanthus bellidiformis. The Plant Journal, 2004. 39(3): p. 319-333.
61. Shao, H., et al., Crystal structures of a multifunctional triterpene/flavonoid glycosyltransferase from Medicago truncatula. Plant Cell, 2005. 17(11): p. 3141-54.
62. He, X. Z., X. Wang, and R. A. Dixon, Mutational analysis of the Medicago glycosyltransferase UGT71G1 reveals residues that control regioselectivity for (iso)flavonoid glycosylation. J Biol Chem, 2006. 281(45): p. 34441-7.
63. Offen, W., et al., Structure of a flavonoid glucosyltransferase reveals the basis for plant natural product modification. The EMBO Journal, 2006. 25(6): p. 1396-1405.
64. Masada, S., K. Terasaka, and H. Mizukami, A single amino acid in the PSPG-box plays an important role in the catalytic function of CaUGT2 (Curcumin glucosyltransferase), a Group D Family 1 glucosyltransferase from Catharanthus roseus. FEBS Letters, 2007. 581(14): p. 2605-2610.
65. Altschul, S., Gish, W., Miller, W., Myers, E., and Lipman, D. (1990). Basic local alignment search tool. Journal of Molecular Biology. 215 (3): 403-4.
66. Henikoff, S. and J. G. Henikoff (1992). “Amino acid substitution matrices from protein blocks.” Proceedings of the National Academy of Sciences of the United States of America 89(22): 10915-10919.

### INCORPORATION BY REFERENCE; EQUIVALENTS

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1. A compound represented by the following structural formula: 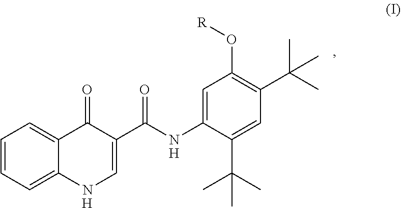
or a pharmaceutically acceptable salt thereof,
wherein R is a monosaccharide, a disaccharide, a trisaccharide, or an oligosaccharide comprising 4 to 10 monosaccharides.

2. The compound of claim 1, wherein R is a monosaccharide.

3. The compound of claim 2, wherein the monosaccharide is a pentose monosaccharide, hexose monosaccharide, or heptose monosaccharide.

4. The compound of claim 1, wherein R is allose, apiose, arabinose, fructose, fucitol, fucose, galactose, glucose, glucuronic acid, mannose, N-acetylglucosamine, N-acetylgalactosamine, rhamnose, or xylose.

5. The compound of claim 1, wherein R is glucosamine, galactosamine, mannosamine, 5-thio-D-glucose, nojirimycin, deoxynojirimycin, 1,5-anhydro-D-sorbitol, 2,5-anhydro-D-mannitol, 2-deoxy-D-galactose, 2-deoxy-D-glucose, 3-deoxy-D-glucose, arabinitol, galactitol, glucitol, iditol, lyxose, mannitol, L-rhamnitol, 2-deoxy-D-ribose, ribose, ribitol, ribulose, xylulose, altrose, gulose, idose, levulose, psicose, sorbose, tagatose, talose, galactal, glucal, fucal, rhamnal, arabinal, xylal, 3,4-di-O-acetyl-L-fucal, 3,4-di-O-acetyl-L-rhamnal, 3,4-di-O-acetyl-D-arabinal, 3,4-di-O-acetyl-D-xylal, valienamine, validamine, valiolamine, valienol, valienone, galacturonic acid, mannuronic acid, N-acetylneuraminic acid, N-acetylmuramic acid, gluconic acid D-lactone, galactonic acid gamma-lactone, galactonic acid delta-lactone, mannonic acid gamma-lactone, D-altro-heptulose, D-manno-heptulose, D-glycero-D-manno-heptose, D-glycero-D-gluco-heptose, D-allo-heptulose, D-altro-3-heptulose, D-glycero-D-manno-heptitol, or D-glycero-D-altro-heptitol.

6. The compound of claim 1, wherein R is a disaccharide.

7. The compound of claim 6, wherein R is a disaccharide of two glucose molecules.

8. The compound of claim 6, wherein R is a disaccharide of two galactose molecules.

9. The compound of claim 6, wherein R is a disaccharide of two xylose molecules.

10. The compound of claim 6, wherein the disaccharide molecules are bonded by a 1→2 glycosidic bond.

11. The compound of claim 1, wherein R is a trisaccharide.

12. The compound of claim 11, wherein R is a trisaccharide of three glucose molecules.

13. The compound of claim 11, wherein R is a trisaccharide of three galactose molecules.

14. The compound of claim 11, wherein R is a trisaccharide of three xylose molecules.

15. The compound of claim 11, wherein the trisaccharide molecules are bonded by a 1→2 glycosidic bond and by a 1→4 glycosidic bond.

16. (canceled)

17. A method of making an ivacaftor glycoside, the method comprising:
a) providing a reaction mixture comprising:
i) a compound having the following structural formula: 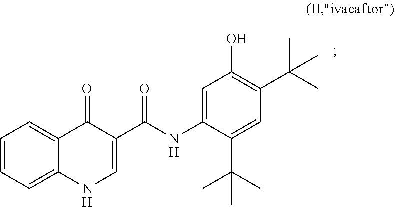
ii) a uridine diphosphate glycosyltransferase (UGT); and
iii) uridine diphosphate-monosaccharide;
b) allowing the reaction mixture to convert ivacaftor to a monosaccharide, a disaccharide, a trisaccharide, or an oligosaccharide of ivacaftor.

18. The method of claim 17, wherein the UGT comprises an amino acid sequence that is at least 95% similar to SEQ ID NO: 1.

19. The method of claim 17, wherein the UGT comprises an amino acid sequence that is at least 80% similar to a region from V278 to Q318 of SEQ ID NO: 1.

20. The method of claim 17, wherein the UGT comprises an amino acid sequence that is:
a) at least 90% similar to a region from 167 to D75 of SEQ ID NO: 1;
b) at least 90% similar to a region from D106 to L114 of SEQ ID NO: 1;
c) at least 90% similar to a region from C127 to S129 of SEQ ID NO: 1; and
d) at least 80% similar to a region from V278 to Q318 of SEQ ID NO: 1.

21. The method of claim 17, wherein the UGT comprises an amino acid sequence that is at least 95% similar to SEQ ID NO: 2.

22. The method of claim 17, wherein the UGT comprises an amino acid sequence that is at least 80% similar to a region from V287 to Q327 of SEQ ID NO: 2.

23. The method of claim 17, wherein the UGT comprises an amino acid sequence that is:
a) at least 90% similar to a region from 163 to G70 of SEQ ID NO: 2;
b) at least 90% similar to a region from D106 to 1114 of SEQ ID NO: 2;
c) at least 90% similar to a region from C127 to T129 of SEQ ID NO: 2; and
d) at least 80% similar to a region from V287 to Q327 of SEQ ID NO: 2.

24. The method of claim 17, wherein the UGT comprises an amino acid sequence that is at least 95% similar to SEQ ID NO: 3.

25. The method of claim 17, wherein the UGT comprises an amino acid sequence that is at least 80% similar to a region from V280 to Q320 of SEQ ID NO: 3.

26. The method of claim 17, wherein the UGT comprises an amino acid sequence that is:
a) at least 90% similar to a region from 167 to D75 of SEQ ID NO: 3;
b) at least 90% similar to a region from D106 to L114 of SEQ ID NO: 3;
c) at least 90% similar to a region from C127 to S129 of SEQ ID NO: 3; and
d) at least 80% similar to a region from V280 to Q320 of SEQ ID NO: 3.

27. The method of claim 17, wherein the UGT comprises an amino acid sequence that is at least 95% similar to SEQ ID NO: 4.

28. The method of claim 17, wherein the UGT comprises an amino acid sequence that is at least 80% identical to a region from V283 to Q323 of SEQ ID NO: 4.

29. The method of claim 17, wherein the UGT comprises an amino acid sequence that is:
a) at least 90% similar to a region from 167 to Q79 of SEQ ID NO: 4;
b) at least 90% similar to a region from D110 to L118 of SEQ ID NO: 4;
c) at least 90% similar to a region from C131 to T133 of SEQ ID NO: 4; and
d) at least 80% similar to a region from V283 to Q323 of SEQ ID NO: 4.

30. The method of claim 17, wherein the uridine diphosphate-monosaccharide is uridine diphosphate-glucose (“UDP-glucose”).

31. The method of claim 17, wherein the uridine diphosphate-monosaccharide is uridine diphosphate-galactose (“UDP-galactose”).

32. The method of claim 17, wherein the uridine diphosphate-monosaccharide is uridine diphosphate-xylose (“UDP-xylose”).

33. The method of claim 17, wherein the uridine diphosphate-monosaccharide is uridine diphosphate-N-acetylglucosamine (“UDP-N-acetylglucosamine”).

34. A method of treating cystic fibrosis or a cystic fibrosis transmembrane conductance regulator related disease, the method comprising administering to a patient in need thereof a therapeutically effective amount of a compound having the following structural formula: 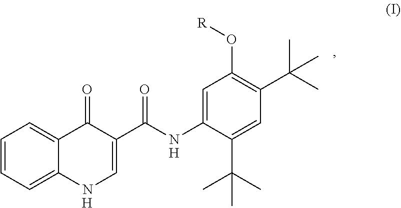
or a pharmaceutically acceptable salt thereof,
wherein R is a monosaccharide, a disaccharide, a trisaccharide, or an oligosaccharide comprising 4 to 10 monosaccharides.

35-37. (canceled)
